SEQUENCE LISTING (58 sequences)

SEQ ID NO: 1 - protein, 4 aa, Artificial Sequence
  Description of Artificial Sequence Synthetic peptide
HHKK

SEQ ID NO: 2 - RNA, 25 nt, Artificial Sequence
  Description of Artificial Sequence Synthetic oligonucleotide
cacaacaaaugugaaugcagaccaa

SEQ ID NO: 3 - RNA, 25 nt, Artificial Sequence
  Description of Artificial Sequence Synthetic oligonucleotide
uuggucugcauucacauuuguugug

SEQ ID NO: 4 - DNA, 21 bp, Artificial Sequence
  Description of Artificial Sequence Synthetic oligonucleotide
ucgagacccugguggacautt

SEQ ID NO: 5 - DNA, 21 bp, Artificial Sequence
  Description of Artificial Sequence Synthetic oligonucleotide
auguccaccagggucucgatt

SEQ ID NO: 6 - RNA, 25 nt, Artificial Sequence
  Description of Artificial Sequence Synthetic oligonucleotide
cuguagacacacccacccacauaca

SEQ ID NO: 7 - RNA, 25 nt, Artificial Sequence
  Description of Artificial Sequence Synthetic oligonucleotide
gacuuccugaccuuggagcaucuca

SEQ ID NO: 8 - RNA, 25 nt, Artificial Sequence
  Description of Artificial Sequence Synthetic oligonucleotide
gaggucacccgcgugcuaauggugg

SEQ ID NO: 9 - protein, 17 aa, Artificial Sequence
  Description of Artificial Sequence Synthetic peptide
KHHHKHHHKHHHKHHHK

SEQ ID NO: 10 - protein, 17 aa, Artificial Sequence
  Description of Artificial Sequence Synthetic peptide
KHHHKHHHHKHHHKHHH

SEQ ID NO: 11 - DNA, 30 bp, Artificial Sequence
  Description of Artificial Sequence Synthetic primer
gaacatcgatgacaagcttaggtatcgata

SEQ ID NO: 12 - DNA, 47 bp, Artificial Sequence
  Description of Artificial Sequence Synthetic primer
gaacatcgatgacaagcttaggtatcgatacaagctgcctcgccttg

SEQ ID NO: 13 - DNA, 30 bp, Artificial Sequence
  Description of Artificial Sequence Synthetic primer
gatgtctaccagcgaagctactgccgtccg

SEQ ID NO: 14 - DNA, 47 bp, Artificial Sequence
  Description of Artificial Sequence Synthetic primer
gaacatcgatgacaagcttaggtatcgataggtcactgacagaggcg

SEQ ID NO: 15 - DNA, 30 bp, Artificial Sequence
  Description of Artificial Sequence Synthetic primer
ggcgctgctagctgtcgctctgtggttctg

SEQ ID NO: 16 - RNA, 25 nt, Artificial Sequence
  Description of Artificial Sequence Synthetic oligonucleotide
ccaugccaaguggucccaggcugca

SEQ ID NO: 17 - RNA, 25 nt, Artificial Sequence
  Description of Artificial Sequence Synthetic oligonucleotide
ugcagccugggaccacuuggcaugg

SEQ ID NO: 18 - RNA, 25 nt, Artificial Sequence
  Description of Artificial Sequence Synthetic oligonucleotide
ccaacaucaccaugcagauuaugcg

SEQ ID NO: 19 - RNA, 25 nt, Artificial Sequence
  Description of Artificial Sequence Synthetic oligonucleotide
cgcauaaucugcauggugauguugg

SEQ ID NO: 20 - RNA, 25 nt, Artificial Sequence
  Description of Artificial Sequence Synthetic oligonucleotide
uguaugugggugggugugucuacag

SEQ ID NO: 21 - RNA, 25 nt, Artificial Sequence
  Description of Artificial Sequence Synthetic oligonucleotide
cacuuuggguccggagggcgagacu

SEQ ID NO: 22 - RNA, 25 nt, Artificial Sequence
  Description of Artificial Sequence Synthetic oligonucleotide
agucucgcccuccggacccaaagug

SEQ ID NO: 23 - RNA, 25 nt, Artificial Sequence
  Description of Artificial Sequence Synthetic oligonucleotide
ccugaugagaucgaguacaucuuca

SEQ ID NO: 24 - RNA, 25 nt, Artificial Sequence
  Description of Artificial Sequence Synthetic oligonucleotide
ugaagauguacucgaucucaucagg

SEQ ID NO: 25 - RNA, 25 nt, Artificial Sequence
  Description of Artificial Sequence Synthetic oligonucleotide
gagagaugagcuuccuacagcacaa

SEQ ID NO: 26 - RNA, 25 nt, Artificial Sequence
  Description of Artificial Sequence Synthetic oligonucleotide
uugugcuguaggaagcucaucucuc

SEQ ID NO: 27 - RNA, 25 nt, Artificial Sequence
  Description of Artificial Sequence Synthetic oligonucleotide
gcaaggcgaggcagcuugaguuaaa

SEQ ID NO: 28 - RNA, 25 nt, Artificial Sequence
  Description of Artificial Sequence Synthetic oligonucleotide
uuuaacucaagcugccucgccuugc

SEQ ID NO: 29 - RNA, 25 nt, Artificial Sequence
  Description of Artificial Sequence Synthetic oligonucleotide
ccucuucuguaagacacucacaauu

SEQ ID NO: 30 - RNA, 25 nt, Artificial Sequence
  Description of Artificial Sequence Synthetic oligonucleotide
aauugugagugucuuacagaagagg

SEQ ID NO: 31 - RNA, 25 nt, Artificial Sequence
  Description of Artificial Sequence Synthetic oligonucleotide
cccuugaguccaaucacacaauuaa

SEQ ID NO: 32 - RNA, 25 nt, Artificial Sequence
  Description of Artificial Sequence Synthetic oligonucleotide
uuaauugugugauuggacucaaggg

SEQ ID NO: 33 - RNA, 25 nt, Artificial Sequence
  Description of Artificial Sequence Synthetic oligonucleotide
ccaagugauugaagcagaugccuuu

SEQ ID NO: 34 - RNA, 25 nt, Artificial Sequence
  Description of Artificial Sequence Synthetic oligonucleotide
aaaggcaucugcuucaaucacuugg

SEQ ID NO: 35 - RNA, 25 nt, Artificial Sequence
  Description of Artificial Sequence Synthetic oligonucleotide
gagcauggaagaggauucuggacuc

SEQ ID NO: 36 - RNA, 25 nt, Artificial Sequence
  Description of Artificial Sequence Synthetic oligonucleotide
gaguccagaauccucuuccaugcuc

SEQ ID NO: 37 - RNA, 25 nt, Artificial Sequence
  Description of Artificial Sequence Synthetic oligonucleotide
cauggaagaggauucuggacucucu

SEQ ID NO: 38 - RNA, 25 nt, Artificial Sequence
  Description of Artificial Sequence Synthetic oligonucleotide
agagaguccagaauccucuuccaug

SEQ ID NO: 39 - RNA, 25 nt, Artificial Sequence
  Description of Artificial Sequence Synthetic oligonucleotide
ccugaccuuggagcaucucaucugu

SEQ ID NO: 40 - RNA, 25 nt, Artificial Sequence
  Description of Artificial Sequence Synthetic oligonucleotide
acagaugagaugcuccaaggucagg

SEQ ID NO: 41 - RNA, 25 nt, Artificial Sequence
  Description of Artificial Sequence Synthetic oligonucleotide
gcuaagggcauggaguucuuggcau

SEQ ID NO: 42 - RNA, 25 nt, Artificial Sequence
  Description of Artificial Sequence Synthetic oligonucleotide
augccaagaacuccaugcccuuagc

SEQ ID NO: 43 - RNA, 25 nt, Artificial Sequence
  Description of Artificial Sequence Synthetic oligonucleotide
ugagaugcuccaaggucaggaaguc

SEQ ID NO: 44 - RNA, 25 nt, Artificial Sequence
  Description of Artificial Sequence Synthetic oligonucleotide
ggauccacgagcccaagggcuacca

SEQ ID NO: 45 - RNA, 25 nt, Artificial Sequence
  Description of Artificial Sequence Synthetic oligonucleotide
ugguagcccuugggcucguggaucc

SEQ ID NO: 46 - RNA, 25 nt, Artificial Sequence
  Description of Artificial Sequence Synthetic oligonucleotide
cccaagggcuaccaugccaacuucu

SEQ ID NO: 47 - RNA, 25 nt, Artificial Sequence
  Description of Artificial Sequence Synthetic oligonucleotide
agaaguuggcaugguagcccuuggg

SEQ ID NO: 48 - RNA, 25 nt, Artificial Sequence
  Description of Artificial Sequence Synthetic oligonucleotide
gagcccaagggcuaccaugccaacu

SEQ ID NO: 49 - RNA, 25 nt, Artificial Sequence
  Description of Artificial Sequence Synthetic oligonucleotide
aguuggcaugguagcccuugggcuc

SEQ ID NO: 50 - RNA, 25 nt, Artificial Sequence
  Description of Artificial Sequence Synthetic oligonucleotide
gauccacgagcccaagggcuaccau

SEQ ID NO: 51 - RNA, 25 nt, Artificial Sequence
  Description of Artificial Sequence Synthetic oligonucleotide
augguagcccuugggcucguggauc

SEQ ID NO: 52 - RNA, 25 nt, Artificial Sequence
  Description of Artificial Sequence Synthetic oligonucleotide
cacgagcccaagggcuaccaugcca

SEQ ID NO: 53 - RNA, 25 nt, Artificial Sequence
  Description of Artificial Sequence Synthetic oligonucleotide
uggcaugguagcccuugggcucgug

SEQ ID NO: 54 - RNA, 25 nt, Artificial Sequence
  Description of Artificial Sequence Synthetic oligonucleotide
ccaccauuagcacgcgggugaccuc

SEQ ID NO: 55 - RNA, 25 nt, Artificial Sequence
  Description of Artificial Sequence Synthetic oligonucleotide
guacaacagcacccgcgaccgggug

SEQ ID NO: 56 - RNA, 25 nt, Artificial Sequence
  Description of Artificial Sequence Synthetic oligonucleotide
cacccggucgcgggugcucuucuac

SEQ ID NO: 57 - RNA, 25 nt, Artificial Sequence
  Description of Artificial Sequence Synthetic oligonucleotide
guggauccacgagcccaagggcuac

SEQ ID NO: 58 - RNA, 25 nt, Artificial Sequence
  Description of Artificial Sequence Synthetic oligonucleotide
guagcccuugggcucguggauccag

(19) United States
(12) Patent Application Publication
(10) Pub. No.: US 2013/0123330 A1
(43) Pub. Date: May 16, 2013

(54) Dual Targeted siRNA Therapeutics for Treatment of Diabetic Retinopathy and Other Ocular Neovascularization Diseases

(71) Applicants: Patrick Y. Lu, Rockville, MD (US); John J. Xu, Germantown, MD (US); Alan Y. Lu, Baltimore, MD (US)

(72) Inventors: Patrick Y. Lu, Rockville, MD (US); John J. Xu, Germantown, MD (US); Alan Y. Lu, Baltimore, MD (US)

(21) Appl. No.: 13/549,514

(22) Filed: Jul. 15, 2012

Related U.S. Application Data
(60) Provisional application No. 61/508,593, filed on Jul. 15, 2011.

(51) Int. Cl. A61K 31/713

(52) U.S. Cl. CPC A61K 31/713
USPC 514/44 A

(57) ABSTRACT

The present invention relates to compositions and methods for treating diabetic retinopathy and other ocular neovascularization diseases. In one embodiment, the composition comprises at least two different siRNA duplexes and a pharmaceutically acceptable carrier. One of the duplexes binds to an mRNA molecule that encodes VEGF, and the other binds to an mRNA molecule that encodes VEGFR2. In another embodiment, the composition further comprises an siRNA duplex that binds to an mRNA molecule that encodes TGFβ1.

### CROSS-REFERENCE TO RELATED PATENT APPLICATIONS

This application claims the benefit of and priority to U.S. Provisional Patent Application No. 61/508,593, filed Jul. 15, 2011, which is incorporated herein by reference in its entirety.

### FIELD OF INVENTION

The invention relates to siRNA molecules, compositions, and methods for the treatment of diabetic retinopathy and other ocular neovascularization diseases.

### BACKGROUND OF THE INVENTION

Diabetic retinopathy is the most common cause of vision loss in the working-age population around the world. This condition is due to damage in the small blood vessels in retinal tissue—the light-perceiving part of eyes. When these damaged blood vessels begin to leak fluid near the center of the retina, known as the macula, macular edema occurs. The macula provides detailed central vision used for activities such as reading, driving, and distinguishing faces. In macular edema, the retinal tissue swells, which can lead to vision loss if left untreated.

In addition to the diabetic retinopathy, many diverse ocular diseases are the result of excessive neovascularization (NV), an abnormal proliferation and growth of blood vessels within the eye. The development of ocular NV itself has adverse consequences for vision but also is an early pathological step in many serious eye diseases; despite introduction of new therapeutic agents it remains the most common cause of permanent blindness in United States and Europe. Several major eye diseases promote an abnormal neovascularization, which leads to further damage to the eyes causing loss of vision. Unfortunately, few treatment options exist for patients with any of these ocular NV diseases.

The ocular neovascularization diseases can be divided into diseases affecting the anterior, or front, of the eye and those affecting the posterior, or retinal, part of the eye. Development of NV at these different regions may have different origins, but the biochemical and physiological nature of the NV process appears to be virtually identical, regardless of eye region. Consequently, an effective means to intervene in the biochemical nature of ocular NV offers the prospect for providing an effective treatment for any ocular disease that involves ocular NV as the major pathology or as the underlying pathology, regardless of whether the disease afflicts the anterior or posterior of the eye. Nonetheless, the anterior and posterior ocular tissues differ considerably, and these differences can have a dramatic influence on the most effective means to administer therapeutic treatments so that the tissue and cells are reached by the therapeutic agent.

The National Eye Institute of NIH has estimated that 400,000 Americans have had some form of ocular herpes, and there are nearly 50,000 new and recurring cases diagnosed each year in the United States, with the more serious stromal keratitis accounting for about 25%. From a larger study, it was found that the recurrence rate of ocular herpes is 10 percent in one year, 23 percent in two years, and 63 percent within 20 years. Herpes simplex virus (HSV) infection of the eye results in corneal neovascularizaiton, an important step in the blinding immunopathological lesion of stromal keratitis (SK). Although application of available anti-viral drugs could control the HSV infection to a certain extent, there is no effective medication available that can treat the HSV-caused SK and protect the patients from blindness.

Laser treatment of retina has been the standard care for diabetic macular edema since an NEI-supported study in 1985 showed it to be beneficial. The current anti-angiogenesis targeting therapeutics for treatment of ocular neovascularization diseases are the antagonist inhibitors that block the action of VEGF. The single use of those agents or in combination with laser therapy has demonstrated clinical benefit with improvement of vision acuity. Those agents include Ranibizumab (Lucentis), Pegaptanib sodium (Macugen), and Bevacizumab (Avastin). One major mechanism of action of all those VEGF inhibitors is to block the function of VEGF protein.

Ocular NV Biochemistry and Physiology

Like other tissues, ocular tissues are in a continuous state of maintenance, which often entails neovascularization. This essential process is kept in balance by a balance of pro- and inhibitory factors. Unfortunately, the balance is not correctly maintained in the many ocular neovascularization diseases, and excessive growth of damaging new blood vessels is the result. The process for this excessive neovascularization appears to be virtually identical regardless of the region of the eye and disease, although the originating cause of the pathology as well as the role in vision loss differs widely. The commonality of the pathological process offers means to provide therapeutic interventions that are effective in these diverse diseases of the eye.

The normal cornea is avascular, and HSV does not express any angiogenic protein itself, but infected ocular tissues express angiogenic factors that induce corneal NV. The angiogenic factor production occurs initially from virus-infected corneal epithelial, non-inflammatory, cells followed by expression in a clinical phase from inflammatory cells (PMNs and macrophages) in the stroma. A mouse model of HSV induced corneal NV was developed by implantation of purified HSV viral DNA fragments (HSV DNA, rich in CpG motifs) or synthetic CpG oligonucleotides (CpG ODN). This model is thought to provide a clinically relevant model of corneal NV and herpetic SK disease, and is useful for testing therapeutic modalities for their efficacy in inhibiting ocular NV disease.

An attractive approach for therapeutic intervention is to inhibit the common pathological condition of these diseases. From many studies, it has become established that VEGF-mediated neovascularization and angiogenesis is one of the common pathological pathways of many ocular neovascularization diseases. The VEGF-mediated angiogenesis pathway plays a central role in angiogenesis of all these NV-related eye diseases. The VEGF family is composed of five structurally related growth factors: VEGF-A (VEGF), Placenta Growth factor (PIGF), VEGF-B, VEGF-C, and VEGF-D. Known receptors include three structurally homologous tyrosine kinase receptors, VEGFR-1 (Flt-1), VEGFR-2 (KDR or Flk-1), and VEGFR-3 (Flt-4), with different affinity or functions related to different VEGF members. While function and regulation of four VEGF members are poorly understood, VEGF binds VEGFR-2 and is known to induce neovascularization and angiogenesis, as well as vascular permeability.

This understanding of key players in the VEGF pathway of angiogenesis has led to studies with inhibitors of VEGF as candidate therapeutic agents, including macugen, an aptamer oligonucleotide inhibiting VEGF binding to its receptor. While these studies in ocular angiogenesis, as well as in other angiogenesis diseases such as tumor growth, have validated the value of the VEGF pathway for clinical effect, the experimental agents are far from effective for many patients. It is clear that better inhibitors of the VEGF pathway are needed if we are to develop treatments for these major eye diseases.

VEGFR-2 is up-regulated in proliferating endothelium that may be a direct response to VEGF-A or hypoxia. It is thought that VEGFR-2 is responsible for angiogenic signals for blood vessel growth. VEGFR2 is also known as kinase insert domain receptor (KDR) in humans or fetal liver kinase-1 (Flk-1) in mice, is a member of the class III subfamily of receptor tyrosine kinase (RTKs). VEGFR2 also contains 7 extracellular immunoglobulin-like domains, a membrane-spaning region, and an intracellular tyrosine kinase domain containing a kinase insert sequence. The expression of VEGFR2 is almost exclusively restricted to endothelial cells. The full-length VEGFR2 precursor protein contains 1356 amino acid (aa) with 19 aa signal peptide. VEGFR2 binds VEGF with high affinity results in playing an important role in tumor angiogenesis and other diseases involve pathological angiogenesis. Inactivation of VEGFR2 by a blocking antibody or small molecule TKR inhibitor can disrupt angiogenesis and prevent tumor cell invasion.

There are many studies confirming that VEGF play a central role in neovascularization but can not explain why VEGF antagonists are only partially effective. Recently, Transforming growth factor-beta (TGFβ1) has been implicated in the development of neovascularization (Gerard et al. 2000). Smad 4 plays the most important role in the TGF-β signal transduction (Zimowska 2006). One study has revealed that oxygen-induced retinopathy in neonatal mice is related to the up regulation of TGFβ1 and Smad 4 mRNA expressions in the retina. Several findings suggest that TGFβ is also capable of inducing cellular senescence. For instance, stimulation of human diploid fibroblasts with TGFβ1 triggers the appearance of biomarkers of SIPS such as SA-β-Gal activity and increases mRNA steady state levels of senescence associated genes including Apo J, fibronectin, and SM22. In vitro studies of different cellular systems have shown that TGFβ1 is inducible by oxidative stress. Thus, it has been hypothesized that oxidative stress-induced premature senescence is triggered via an increased expression of TGFβ1. Previous studies have demonstrated that cellular senescence occurs in RPE cells during the aging process in primates. Furthermore, it has been shown in vitro that cellular senescence in human RPE cells is inducible by exposure to mild hyperoxia. Whether human RPE cells undergo senescent changes in AMD is unclear yet. Histochemical studies have detected an increased expression of TGFβ1 in the RPE of patients with AMD. Treatment with neutralizing antibodies against the TGFβ1, prevented the oxidative stress-mediated elevation of senescence-associated biomarkers. On the other hand, TGFβ1 has been revealed as pro-inflammatory factor involving in the tissue scaring which has been suspected as one cause of retina scaring after anti-angiogenesis treatment. Therefore, silencing TGFβ1 in addition to knockdown VEGF pathway factors will likely provide a novel approach for treatment of diabetic retinopathy and AMD.

### BRIEF DESCRIPTION OF THE FIGURES

FIG. 1 Comparison between 25mer siRNA and 21mer siRNA in VEGF gene silencing (A) The most potent 25 mer and 21mer siRNA were selected first from each set of 6 duplexes. Than comparison was carried out with two tumor cell lines expressing human VEGF protein (DLD-1, colon carcinoma and MBA-MD-435, breast carcinoma) using in vitro transfection with Lipo2000 (Invitrogen, CA) followed by RT-PCR analyses. At either 0.3 μg or 2.0 μg doses, 25mer siRNA demonstrated stronger inhibitory activity than 21mer siRNA, especially at 2.0μg. (B) siRNA duplexes with the 25mer blunt-end format and 21mer sticky-end format were compared for their inhibitory effects in MCF-7/VEGF165 cell culture. The expression level of VEGF gene was monitored for 5 days post-transfection. The 25mer blunt-end siRNA duplex was indeed more efficacious than the 21mer.

FIG. 2 Selection of potent siRNA targeting VEGF Eight 25 mer siRNA duplexes targeting VEGF with control siRNA were transfected into human 293 and mouse F3 cells. The Q-RT-PCR reactions were conducted following siRNA transfections to the corresponding cells, with a standard control gene target. The in vitro study has demonstrated that a potent siRNA duplex (hmVEGFc) was selected due to its potent activity for knocking down the target gene within both human and mouse cell.

FIG. 3 Potent siRNAs targeting VEGFR2 gene were identified A web-based siRNA design algorism called BLOCK-IT RNAi Designer is available from Invitrogen for designing 25-mer blunt-ended siRNA. This interactive algorism allows the user to select the siRNA target sequence either using the default parameters or using customized parameters. By inputting the exact target mRNA sequence or GenBanK identification number of target mRNA sequence into the program, one can get a list of up to 10 candidates of 25-mer siRNA sequence with ranking between one-star to five-star. The more ranking stars one siRNA candidate has, the more potent the siRNA duplex is like to have in knocking down target gene expression. We have selected eight 25 mer blunt ended siRNA duplexes with either the BLOCK-IT RNAi Designer or with our own proprietary algorithm. The potent siRNA was selected with transfection of mouse SVR cell followed by total RNA extraction and Q-RT-PCR (A) and Transfection of human HUVEC cell followed by ELISA analysis (B). The eight VEGFR2-siRNA duplexes were transfected with control 25-mer Luc-siRNA (7 ug siRNA/1×10⁶ cells) using electroporation. At 48 hours post transfection, HUVEC cells were harvested and subjected to a hVEGFR2 ELISA assay to measure the concentration of VEGFR2 in cell lysate (3 mg of lysate protein was loaded into each well). Data were presented as Mean+/−STD. The potent 25 mer VEGFR2-siRNA was selected based on the ELISA analysis for future animal tumor model studies. The most potent VEGFR2 specific siRNA duplex (VEGFR2-h) was selected.

FIG. 4. Design and Select the Most Potent siRNA Duplexes Targeting TGFβ1 We used 25 mer blunt-ended siRNA duplexes due their active and durable potencies. Each sequence is able to target both the human and corresponding mouse genes. Before testing those siRNA oligos (synthesized by Qiagen) in the corresponding cells, a human PC-3 cell was used simply because that the three targets expressing from this cell (A). We also surveyed the expressions of TGFβ1 in the mouse C166 cell (B). Eight siRNA duplexes for each targeted were screened by siRNA transfection followed by total RNA isolation and Q-RT-PCR. The most potent siRNA duplex against each target was selected for TGFβ1. The sample normalization was done with a 361 bp fragment of a house keeping gene rig/S15. The selected sequence was listed in the Table 3: hmTF25f.

FIG. 5. Histidine-Lysine Branched Polymer for siRNA Delivery The optimized histidine-lysine polymers (HKP) have been applied for siRNA deliveries in vitro and in vivo. One HK polymer species, H3K4b, having a Lysine backbone with four branches containing multiple repeats of Histidine and Lysine, was used for packaging siRNA with a N/P ratio of 4:1 by mass. The nanoparticles (average size of 150 nm in diameter) were self-assembled as showed in the image of SEM (Scanning Electronic Microscope).

FIG. 6. Characterization of HKP-siRNA Nanoparticles The HKP-siRNA particles were measured with 90Plus Nanoparticle Size Distribution Analyser (Brookheaven Instruments Limited, NY). The results indicated that the average size of this preparation of HKP-siRNA nanoparticle is 159.9 nm in diameter (A), with the Zeta-potential of 38 (B). These measurements are quite consistent with the (SEM) analysis.

FIG. 7. HKP Packaging Improves Subconjunctival siRNA Delivery FITC-labeled siRNA and HKP were self-assembled into the nanoparticle before injected subconjunctivally into mouse eyes for evaluation of siRNA delivery efficiency. Labeled siRNA can be observed in angiogenic corneal cryosection 24 hr after SCJ administration (A), compared to the cryosection form the group treated with naked FITC-labeled siC 1ab through the same route of delivery (B). Two arrows indicate the location of FITC-labeled siRNA.

FIG. 8. Comparisons of anti-angiogenesis activities between local and systemic Anti-angiogenesis activity of siRNA cocktail targeting VEGF, VEGFR1 and VEGFR2 respectively were tested with HKP-mediated local delivery and a different nanoparticle vehicle (LDP) mediated systemic delivery on day 4. The control, naked siRNA and packaged siRNA were tested with either HKP or LDP. N=6. For both (e) and (f), * represents P<0.05 and ** represents P<0.01.

FIG. 9. Distribution of siRNA after Intravitreous Injection A rabbit model was used for evaluation of the siRNA distribution after intravitreous injection of ³H-labeled siRNA. At 24 hours and 72 hours post injection, tissue was collected following euthanasia. The left eye was dissected to collect aqueous fluid, iris, vitreous fluid, retina and sclera (including choroid). The radioactive activities of each tissue was measured by liquid scintillation spectroscopy. The naked siRNA (2 mg) and HKP-siRNA (250μg) were injected. At 72 hours post injection, HKP-siRNA resulted in high level of siRNA counts in Lens and Retina.

FIG. 10. Control Release and Retina Accumulation of HKP-siRNA after Intravitreous Injection (A) the total siRNA recovered from all tissue types at 24 and 72 hour time points. Yellow bar represents the counts from the naked-siRNA and blue bar represents HKP-siRNA which shows much high recovery rate at both time points. (B) the siRNA counts in retina tissue. Red bar represents the counts of naked-siRNA and green bar represents the HKP-siRNA which shows accumulated siRNA in the retina tissue.

FIG. 11. Comparation of FITC-perfused retinal flatmounts treated with the siRNA cocktail using mouse ROP model Representative FITC-perfused retinal flatmounts on the 17 after hypoxia induced ocular angiogenesis. No. 1 received no treatment; No. 2 was treated with HKP-siRNA_{control} through intravitreous injection; and No. 4 was treated with HKP-siRNA_{control} via subconjunctival injection. No. 5 is a normal control; No. 6 was treated with HKP-siRNA cocktail through intravitreous injection and No. 8 was treated with HKP-siRNA cocktail via subconjunctival injection. No. 6 shows significant improvement regarding the leakage of retinal flatmount.

FIG. 12. Comparation of the representative cryosections treated with the siRNA cocktail using mouse ROP model Representative Cryosections on the 17 after hypoxia induced ocular angiogenesis. No. 1 received no treatment; No. 2 was treated with HKP-siRNA_{control} through intravitreous injection; and No. 4 was treated with HKP-siRNA_{control} via subconjunctival injection. No. 5 is a normal control; No. 6 was treated with HKP-siRNA cocktail through intravitreous injection and No. 8 was treated with HKP-siRNA cocktail via subconjunctival injection. No. 6 shows significant improvement regarding the cryosection imaging with much less angiogenesis staining.

FIG. 13. The target gene knockdown at mRNA level After treatments with the siRNA cocktail packaged with HKP and delivered through intravitreous injection using Hypoxia induced mouse ocular angiogenesis model, the mRNA levels of each target was measured using Q-RT-PCR. The results demonstrated that the specific silence of the target genes, VEGF and VEGFR2, were observed at mRNA level with significant difference from the control groups.

FIG. 14. The target gene knockdown at protein level After treatments with the siRNA cocktail packaged with HKP and delivered through intravitreous injection using Hypoxia induced mouse ocular angiogenesis model, the mRNA levels of each target were measured using ELISA. The results demonstrated that the specific silence of the target genes, VEGF and VEGFR2, were observed at protein level with significant difference from the control groups.

FIG. 15. HKP-mediated TGFβ1 siRNA Delivery Resulted in Speedy Wound Closure The rate of skin wound closure of HKP-TGFβ1 siRNA treated group is significantly higher and the speedy wound closure is result from the gene target silencing.

FIG. 16. HKP-TGFβ1 siRNA Resulted in Less Scar Formation The histological analysis indicated that the structure of the wounded skin tissue after of HKP-TGFβ1 siRNA treatment is very much like the normal skin tissue, in comparison with the wounded skin tissue without treatment. The arrows indicates the scar tissue size in the skin wound area.

FIG. 17. First Generation of Nanoparticle Mediated siRNA Delivery We have developed and collected a series of nanoparticle materials for improving the delivery system for clinically viable siRNA delivery. The system was named as Snano series: Snano 1 is a HKP based system; Snano 2 is a dentrimer based system; Snano 3 is PLGA based system; Snano 4 is small molecular weight of PEG-PEI; Snano 5 is S-DOTAP; and Snano 6 is spermidine based material.

FIG. 18. Chemical Modification of siRNA Oligos In order to stabilize siRNA and minimize the adverse effects of siRNA in vivo (such as off-target effect), certain modification chemistry will be utilized as indicated: Phosphorothioate, Boranophosphate, Methylphosphonate and Phosphodiester. The location of the modification can also be marked as 2-O-Methyl or 2-O-MOE. The structures of those modifications are shown in the figure.

FIG. 19. IND Enabling Study Design and Procedures We have designed a flow chart which is able to reflect the major tasks and procedures for IND enabling studies regarding the HKP-siRNA therapeutics product development for treatment of ocular diseases. The three areas of tasks are (1) HKP-siRNA formulation confirmation; (2) Chemistry, Manufacturing and Controls for API and Excipient; and (3) Pharmacological and Toxicological studies.

FIG. 20. STP601 exhibits potent antiangiogenesis activity with mouse ROP model. A dose dependent response curve with statistical significance (P<0.05) was observed with treatments of STP601 at 2 μg/μl, 1 μg/μl and 0.5 μg/μl dosages. The therapeutic benefit from STP601 at dosage of 2 μg/μl is better than the Avastin treatment at 25 μg/μl. The negative control siRNA duplexes without any homology to the target genes at dosage of 2 μg/μl also exhibits low level of non-specific inhibitory activity. A combination of VEGF-siRNA and TGFβ-siRNA at dosage of 2 μg/μl also exhibits the anti-angiogenesis activity but not statistically significant.

### DESCRIPTION OF THE INVENTION

The present invention provides compositions and methods for treatment of eye diseases, such as diabetic retinopathy (DR), age related macular degeneration (AMD), uveitis, and herpetic stromal keratitis (SK). In one embodiment, the invention uses RNAi-mediated inhibition of gene expression and biochemical pathways to achieve therapeutic benefits for eye diseases. The invention provides RNAi agents, including a cocktail of siRNA oligonucleotides (a pair or triple of oligos), to inhibit 1) VEGF and VEGF receptor 2, and 2) pro-inflammatory factor TGFβ1. In one aspect of this embodiment, the siRNA oligo is specific to VEGF mRNA of human, mouse, and non-human primates. In another aspect of this embodiment, the siRNA oligo is specific to VEGF receptor 2 (VEGFR2) mRNA of human, mouse, and non-human primates. In a third aspect of this embodiment, the siRNA oligo is specific to TGFβ1 mRNA of human, mouse, and non-human primates. In another embodiment, the invention uses locally administered, chemically synthesized carriers that provide delivery of synthetic siRNA oligonucleotides. The methods include using combinations of siRNAs with the dual targeting property as the active pharmaceutical ingredient (API), using peptide polymers as the drug carrier (excipient), and the process for making clinically viable siRNA-peptide polymer nanoparticle formulations for treatment of the ocular diseases. In another embodiment, a histidine-lysine branched peptide (HKP) is used as an siRNA carrier for in vivo delivery. In a further embodiment, the self-assembled siRNA-HKP nanoparticle has an improved inhibitory effect through prolong and controlled siRNA release in the disease tissue (cells).

The invention provides for siRNA-mediated anti-angiogenic effects localized at ocular tissues and at tissues with neovascularization disease. The invention provides methods for using nucleic acid and peptides agents, small molecules, monoclonal antibodies, and aptamers to inhibit excessive neovascularization in eye diseases. The invention also provides for combinations of agents that provide inhibition of the multiple factors and the multiple biochemical pathways that induce unwanted ocular neovascularization. The invention further provides clinical means for delivery of therapeutic agents to ocular tissues (e.g., intra-vitreous and subconjunctival). The methods and compositions of the invention are useful for treatment of ocular neovascularization resulting from eye infections, diabetic retinopathy, age-related macular degeneration, and eye cancer. In still another embodiment, the compositions of the invention result in a further therapeutic benefit when used with another anti-angiogenic agent, such as a monoclonal antibody (e.g., Lucentis), small molecule drug, aptamer drug, or anti-miRNA drug.

The present invention relates to a composition comprising at least two different siRNA duplexes and a pharmaceutically acceptable carrier. One of the duplexes binds to an mRNA molecule that encodes VEGF, and the other binds to an mRNA molecule that encodes VEGFR2. In one embodiment, the composition further comprises an siRNA duplex that binds to an mRNA molecule that encodes TGFβ1. In one aspect of these embodiments, the duplexes target both human mRNA and the homologous mouse mRNA.

As used herein, an “siRNA duplex,” an “siRNA molecule,” or an “RNAi agent” is a duplex RNA oligonucleotide, that is a short, double-stranded RNA molecule, that interferes with the expression of a gene in a cell that produces RNA, after the molecule is introduced into the cell. For example, it targets and binds to a complementary nucleotide sequence in a single stranded (ss) target RNA molecule, such as an mRNA or a micro RNA (miRNA). The target RNA is then degraded by the cell. Such molecules are constructed by techniques known to those skilled in the art. Such techniques are described in U.S. Pat. Nos. 5,898,031, 6,107,094, 6,506,559, and 7,056,704 and in European Pat. Nos. 1214945 and 1230375, which are incorporated herein by reference in their entireties.

As used herein, the singular forms “a,” “an,” and “the” refer to one or more, unless the context clearly indicates otherwise.

In one embodiment, the siRNA duplex is a double-stranded oligonucleotide with a length of about 16 to about 35 base pairs. In one aspect of this embodiment, the duplex is a double-stranded oligonucleotide with a length of about 16 to about 27 base pairs. In another aspect of this embodiment, it is a double-stranded oligonucleotide with a length of about 21 to about 25 base pairs. In still another aspect of this embodiment, it is a double-stranded oligonucleotide with a length of about 25 base pairs. In all of these aspects, the molecule may have blunt ends at both ends, or sticky ends at both ends, or a blunt end at one end and a sticky end at the other.

The siRNA duplexes can be made of naturally occurring ribonucleotides, i.e., those found in living cells, or one or more of its nucleotides can be chemically modified by techniques known in the art. In addition to being modified at the level of one or more of its individual nucleotides, the backbone of the oligonucleotide can be modified. Additional modifications include the use of small molecules (e.g. sugar molecules), amino acid molecules, peptides, cholesterol, and other large molecules for conjugation onto the siRNA molecule.

The siRNA duplexes are any ones that bind to mRNA that encodes VEGF, mRNA that encodes VEGFR2, and/or mRNA that encodes TGFβ1 as the case may be. The duplexes can produce additive or synergistic effects in the cells, depending on the compositions and structures of the particular molecules. In one embodiment, the duplexes are selected from the ones listed in Tables 1-3.

The siRNA duplexes of the invention also include ones derived from those listed in Tables 1-3. The derived molecules can have less than the 25 base pairs shown for each duplex, down to 16 base pairs, so long as the “core” base pairs remain. That is, once given the specific sequences shown in the tables, a person skilled in the art can synthesize duplexes that, in effect, “remove” one or more base pairs from either or both ends in any order, leaving the remaining contiguous base pairs, creating shorter duplexes that are 24, 23, 22, 21, 20, 19, 18, 17, or 16 base pairs in length. Thus, the derived duplexes consist of: a) 24 contiguous base pairs of any one or more of the duplexes in Tables 1-3; b) 23 contiguous base pairs of any one or more of the duplexes in Tables 1-3; c) 22 contiguous base pairs of any one or more of the duplexes in Tables 1-3; b) 21 contiguous base pairs of any one or more of the duplexes in Tables 1-3; d) 20 contiguous base pairs of any one or more of the duplexes in Tables 1-3; e) 19 contiguous base pairs of any one or more of the duplexes in Tables 1-3; f) 18 contiguous base pairs of any one or more of the duplexes in Tables 1-3; g) 17 contiguous base pairs of any one or more of the duplexes in Tables 1-3; and h) 16 contiguous base pairs of any one or more of the duplexes in Tables 1-3. It is not expected that duplexes shorter than 16 base pairs would have sufficient activity or sufficiently low off-target effects to be pharmaceutically useful; however, if any such constructs did, they would be equivalents within the scope of this invention.

Alternatively, the derived duplexes can have more than the 25 base pairs shown for each duplex, so long as the “core” base pairs remain. That is, once given the specific sequences shown in the tables, a person skilled in the art can synthesize duplexes that, in effect, “add” one or more base pairs to either or both ends in any order, creating duplexes that are 26 or more base pairs in length and containing the original 25 contiguous base pairs.

The compositions of the invention can include one or more additional compounds or compositions that inhibit ocular neovascularization. Such compounds and compositions are known to those skilled in the art. They include, for example, Ranibizumab (Lucentis), Pegaptanib sodium (Macugen), and Bevacizumab (Avastin).

The pharmaceutically acceptable carrier is a branched peptide, a polymer, a lipid, or a micelle. Such carriers include a polycationic binding agent, cationic lipid, cationic micelle, cationic polypeptide, hydrophilic polymer grafted polymer, non-natural cationic polymer, cationic polyacetal, hydrophilic polymer grafted polyacetal, ligand functionalized cationic polymer, and ligand functionalized-hydrophilic polymer grafted polymer. In one embodiment, the carrier comprises a histidine-lysine co-polymer that forms a nanoparticle containing the siRNA duplexes. The size of the nanoparticle is 100-400 nm in diameter. More than one type of carrier can be used.

The claimed compositions are useful for treating ocular disease in a subject, where such disease is characterized at least in part by neovascularization. A therapeutically effective amount of the composition is administered to the subject. The dosages, methods, and times administration are readily determinable by persons skilled in the art, given the teachings contained herein. The siRNA duplexes bind to the intended target mRNAs in the ocular cells of the subject.

The term “subject” refers to any animal, including humans, other primates, veterinary animals, such as horses, pigs, goats, cattle, dogs, cats, and sheep, and rodents, such as a mouse, rat, or guinea pig. Rodents are particularly useful for laboratory experiments. In one embodiment, the animal is a mammal. In one particular embodiment, the mammal is a human patient.

Ocular diseases treatable with the compositions of the invention include diabetic retinopathy, macular edema, herpetic stromal keratits, age-related macular degeneration, uveitis, rubeosis, conjunctivitis, keratitis, and iritis. In one embodiment, the disease is proliferative diabetic retinopathy, macular edema, or age-related macular degeneration.

The compositions may the administered directly to the eye, such as topically, subconjunctivally, or intravitreally, or they may be administered at a site distal to the eye, such as intravenously or subcutaneously.

The invention includes a method for identifying the desired siRNA molecules comprising the steps of: (a) creating a collection of siRNA molecules designed to target a complementary nucleotide sequence in the target mRNA molecules, wherein the targeting strands of the siRNA molecules comprise various sequences of nucleotides; (b) selecting the siRNA molecules that show the highest desired effect against the target mRNA molecules in vitro; (c) evaluating the selected siRNA molecules in an animal model or models; and (d) selecting the siRNA molecules that show the greatest efficacy in the model. In one embodiment, the method further includes the steps of adding a pharmaceutically acceptable carrier to each of the siRNA molecules selected by step (b) to form pharmaceutical compositions and evaluating each of the pharmaceutical compositions in the animal model or models.

In an alternative embodiment, the siRNA molecules are examined in an in vitro organ culture assay for their silencing activity and therapeutic efficacy.

In one embodiment, the siRNA sequences are prepared in such way that each one can target and inhibit the same gene from, at least, both human and mouse, or human and non-human primate, or human, mouse, and non-human primate. In one aspect, the siRNA molecules bind to both a human mRNA molecule and a homologous mouse mRNA molecule. That is, the human and mouse mRNA molecules encode proteins that are substantially the same in structure or function. Therefore, the efficacy and toxicity reactions observed in mouse models provide a good understanding about what is going to happen in humans. The selected siRNA molecules are good candidates for human pharmaceutical agents.

### Experimental Design and Techniques

VEGF is an essential growth factor responsible for normal vasculogenesis and angiogenic remodeling. Under some disease conditions VEGF angiogenic pathway will be activated, such as in the situation of tumors where new blood vessels are formed to deliver enough oxygen and nutrition to the rapidly growing abnormal tissues. The majority of severe visual loss in the United States results from complications associated with retinal neovascularization in patients with ischemic ocular disease such as diabetic retinopathy, retinal vein occlusion, and retinopathy of prematurity. Intraocular expression of the angiogenic protein VEGF is closely correlated with neovascularization in these human disorders and with ischemia-induced retinal neovascularization in mice. Therefore, the VEGF pathway, composed of VEGFs and VEGF receptors, is a logical target for inhibition of retinal angiogenesis.

To evaluate anti-angiogenic agents for development of novel therapeutics for ocular NV diseases, published clinically relevant animal models are available. One clinically relevant model for retinal angiogenesis uses hypoxia to induce excessive angiogenesis. Another model for retinal angiogenesis uses laser burns to create lesions in the retina into which angiogenesis occurs. One clinically relevant model for corneal NV uses disease induction either by CpG previously implanted in the micropocket in mice cornea stroma or HSV infection, and through which the inhibition of angiogenesis can easily be measured. The effects of candidate therapeutics can be first tested in cell culture and then selected for studies in clinically relevant animal models of disease.

### RNAi Therapeutic Approach

In RNA interference (RNAi), a double stranded RNA oligo is able to facilitate a sequence-specific degradation of messenger RNA (mRNA), often called gene silencing. This powerful method has been proven to be an important tool for gene function discovery and validation, and it holds great potential in developing novel gene-specific drugs. In our anti-angiogenic RNAi design for the inhibition of eye NV, VEGF and VEGFR2 are chosen since they are key players in the VEGF angiogenic pathway. Small interfering RNAs (siRNAs) have been designed according to general guidelines proposed by Tuchl's research team. The siRNAs are 21-nucleotide long double stranded RNAs with 2-nt overhangs at either 3′ termini, with the negative strand complementary to the targeted mRNA sequences. The knockdown of these genes, singly or in combination, has the impact of blocking the angiogenic pathway leading to the inhibition of NV, and thus the relief of the SK symptoms. The same methodology applies to other NV-related ocular diseases.

A significant need exists for nucleic acid delivery systems for RNAi agents for ocular neovascularization diseases. Understanding the mechanisms of RNAi and its rapidly expanding application represents a major breakthrough during the last decade in the field of biomedicine. Use of siRNA duplexes to interfere with expression of a specific gene requires knowledge of target accessibility, but is blocked by a lack of effective delivery of the siRNA into the target cells for ocular NV diseases. Along with the fast growing literature on siRNA as a functional genomics tool, there is emerging interest in using siRNA as a novel therapeutic. Today, more than two dozens clinical investigations are ongoing. Therapeutic applications clearly depend upon effective local and systemic delivery methods. The advantages of using siRNA as a therapeutic agent are due to its specificity, stability, potency, natural mechanism of action, and uniform chemical nature of agents targeting different gene targets since they differ only in nucleotide sequence.

We have used a polypeptide-based carrier known as Histidine-Lysine Polymer (HKP), to deliver siRNAs in vitro and in vivo. This technology (see WO 0147496, the contents of which are hereby incorporated by reference in their entirety) is able to substantially reduce the formation of neovasculature induced by CpG and Herpes Virus induced corneal angiogenesis with a mouse model, and a hypoxia induced retina angiogenesis with a mouse model. For evaluation of the ocular tissue distribution, a Dutch-Belted rabbit model was used. Our success in the siRNA design and experimentation exhibit the great possibility of developing RNAi therapeutics to cure diabetic retinopathy such as diabetic macular edema, age-related macular degenerations, herpetic stroma keratitis, and other angiogenic eye diseases (10).

### RNAi and Therapeutic Agents

RNAi is a potent method that can be used to knock down gene expression, destroying an mRNA in a sequence-specific manner. RNAi can be managed to provide biological function in a rapid and sustained fashion. The present invention provides RNAi agents giving a gene selective intervention to treat ocular NV or other NV-related eye diseases, as a means to control human eye diseases.

The invention provides siRNA agents that inhibit VEGF and VEGFR2 gene expression and intervene in ocular neovascularization. The invention provides many forms of siRNA molecules as therapeutic agents, including double stranded RNA (dsRNA) oligonucleotides (with or without over hang, sticky or blunt ends), small-hairpin RNA (shRNA), and DNA-derived RNA (ddRNA).

Design of siRNA Sequences

The RNAi agents are designed to have a nucleotide sequence matching a portion of the sequence of a targeted gene. The selected siRNA sequence of the targeted gene may be in any part of the mRNA generated by expression of the gene. The RNAi comprises a sequence that will hybridize with mRNA from the target gene, an “antisense strand” of the siRNA sequence. The siRNA sequence comprises a sequence that will hybridize with the antisense strand, a “sense strand” of the siRNA sequence. The siRNA sequence selected of the targeted gene should not be homologous with any other mRNA generated by the cell, nor with any sequence of the targeted gene that is not transcribed into mRNA. Numerous design rules for selecting a sequence of 20 to 27 bases of the target mRNA sequence are known, including commercially available methods. These design methods evolve and the most current available methods can be used. Designs can be obtained from at least three methods and a single consensus list of highest priority constructed and assembled from these methods. We have found that preparation of at least 6 of the highest priority candidate sequences, followed by cell culture testing for gene inhibition nearly always reveals at least two active siRNA sequences. If not, a second round of obtaining six highest priority candidate sequences and testing can be used.

Besides identification of active siRNA sequences, the design also must ensure homology only with the target mRNA sequences. A poor homology of siRNA sequences with genomic sequences other than those of the target gene mRNA reduces off-target effects at either the mRNA level or the gene level. Also, a poor homology of the “sense strand” of the siRNA sequence reduces off-target effects. By DNA comparison with Clone Manager Suite and by on-line Blast search, the targeted sequences of the selected gene can be confirmed to be unique and to lack sequence homology for other genes including human counterparts. For example, sequences matching the mRNA of mVEGF-A are confirmed to be unique for mVEGF-A without homology for mVEGF-B mRNA, mVEGF-C mRNA, mVEGF-D mRNA, or human counterparts including hVEGF165-a (AF486837). However, the matching sequences will target multiple isoforms of mVEGF-A, e.g., mVEGF (M95200), mVEGF115 (U502791), mVEGF-2 (S38100), mVEGF-A (NM_{—}192823), that encode mVEGF-A proteins of 190 amino acid (aa), 141 aa, 146 aa, and 148 aa, respectively. All of the published cDNA sequences of these mVEGF-A isofoms, except mVEGF-A (NM_{—}192823, a mature form of protein), include a 26-aa signal peptide at the N-terminus. The targeted sequences of mVEGF are chosen not in the signal peptide part, but in the mature protein part shared by all these mVEGF-A isoforms. Targeted sequences of mVEGF-R2 are also confirmed to be unique for these two genes, respectively. Different forms of interfering RNAs are included in present invention. As an example, siRNA sequences are designed according to the above target sequences, using known guidelines. These siRNAs are 25 blunt end stranded RNA oligos (Table 1-3).

The RNAi agents are specific for the target gene sequence, which is dependent upon what species of the organism (animal) we are trying to target. Most mammalian genes share considerable homology, where RNAi agents can be selected to give activity for genes in multiple species with that homologous segment of mRNA of the gene of interest. The preferred siRNA inhibitor design should have perfect homology with both human gene mRNA and a test animal gene mRNA. The test animal(s) should be the one commonly used for efficacy and toxicity studies, such as mouse, rabbit or monkey, in the ocular disease situation.

Since it is known that a minimum of 17-nucleotides (nt) homologous to other gene sequences is required for a siRNA to generate a sequence dependent off-target effects, a blast for each of the 8 possible 17 nt sequences from one 25-mer siRNA may be necessary to investigate the potential of sequence-dependent off-target effect, and use this information as one critical parameter for finalizing the selection of siRNA for API of several siRNA therapeutic programs.

We also checked the siRNA candidates to exclude those containing the known immune stimulatory motif (GU-Rich region, 5′-UGUGU-3′ or 5′-GUCCUUCAA-3′) that may induce the activation of IFN pathway in vivo and in vitro via the TLRs pathway, although our RPP delivery system is highly unlikely to induce the TOLL-like receptor mediated activation of interferon pathway. Finally, we also mapped the targeting region of each tested siRNA sequence to their target mRNA sequences. This mapping is particularly useful for understanding the targeting capability of siRNA candidate on target mRNA and its alternative transcripts.

Clinically Relevant Animal Models

A. A corneal angiogenesis model: In this model, purified HSV DNA (CpG rich) and/or synthetic CpG motif-oligonucleotide (CpG ODN) are used to induce VEGF expression in mouse cornea. This model represents a clinically relevant model of corneal neovascularization with typical characteristics of inflammation-induced angiogenesis and lymphangiogenesis. In this model, the new blood vessel formation is readily induced and measured. Measurements of the angiogenesis areas and HSK disease scores are the most effective ways to evaluate the siRNA-mediated anti-angiogenesis activities in the anterior section of the eyes. The present invention employs this model to test the interfering RNAs and to collect data on the RNAi therapy of the CpG-induced SK, providing quantitative data, and producing an environment close to that of HSV infection-related eye SK in human.

B. An oxygen-induced retinopathy (OIR) model: this mouse model reflects the characters of retinal NV with typical pathogenesis of ischemic and degenerative diseases such as proliferative diabetic retinopathy and age-related macular degeneration. Measurements of the angiogenesis areas through cardiac perfusion with fluorescein-labeled dextran followed by retinal flatmount, cryosection, and mRNA and protein expression levels are applied for evaluation of anti-angiogenesis activity in the posterior section of the eyes.

C. Dutch-Belted rabbit model: This model is used for tissue distribution study after siRNA is administrated through intravitreous injection. Prior to dosing, the animals received an intramuscular sedative injection of a ketamine and xylazine cocktail. The conjunctivas were then flushed with a 1:10,000 solution (equivalent to 0.1 mg/ml) of benzalkonium chloride 50% NF (Spectrum Lab Products, Inc., New Brunswick, N.J.) prepared in 0.9% (w/v) sodium chloride for injection USP (Baxter). A local anesthetic (Alcain, 0.5%) was applied to each eye. For each injection, a new insulin syringe (with pre-fitted needle) was used. Dose formulation was administered by intravitreal injection in both eyes at a dose volume of 50 μl/eye, using a binocular indirect ophthalmoscope to confirm needle placement. The left eye was dissected to collect aqueous fluid, iris, vitreous fluid, retina and sclera (including choroid).

Inhibition of Angiogenic Pathways Mediating Ocular NV

The angiogenesis process, like inflammation, is complex but highly conserved across tissues. Another similarity is the major role several secreted factors play. An early step is driven by the VEGF pathway that involves secretion of VEGF growth factors, which bind and activate cells bearing different members of the VEGF family of receptors. The invention provides for RNAi agents inhibiting expression of proteins, including transcription factors that now enables therapeutic intervention at these key intracellular steps of neovascularization.

The VEGF family is composed of five structurally related members: VEGF-A, Placenta Growth factor (PIGF), VEGF-B, VEGF-C, and VEGF-D. There are three structurally homologous tyrosine kinase receptors in the VEGF receptor family: VEGFR-1 (Flt-1), VEGFR-2 (KDR or Flk-1), and VEGFR-3 (Flt-4), with different affinity or functions related to different VEGF members. While function and regulation of other four VEGF members are less understood, VEGF-A, which binds VEGFR-1 and VEGFR-2, is known to induce neovascularization, angiogenesis, and vascular permeability. In order to functionally interact with their specific receptors VEGF naturally forms homo-dimers. VEGFR-2 is up-regulated in tumor and proliferating endothelium that may be a direct response to VEGF-A or partly to hypoxia. It is well accepted that VEGFR-2 mediates angiogenic signals for blood vessel growth, and is necessary for proliferation. The understanding of basic biology of the VEGF and VEGF receptors provides solid foundation for the design of approaches to target the VEGF signaling pathway. The invention provides RNAi agents specific for inhibition of murine and human forms of VEGF, the VEGF receptors, and intracellular signal transduction pathway.

Inhibition of Ocular NV Endothelial Cell Proliferation

A key step in formation of neovasculature is proliferation of activated endothelial cells in nearby vasculature. This step is an important point for therapeutic intervention. Many intracellular factors are well known to be critical for endothelial cell survival and/or proliferation. The two embodiments provided by the invention are 1) block endothelial cell proliferation and 2) induce apoptosis in activated endothelial cells. Either or both of these embodiments result in a reduction in neovasculature due to inhibition of endothelial cell proliferation and migration. The invention also provides for siRNA initiating activated endothelial cell apoptosis. The nanoparticles provided by the invention deliver the RNAi into the intracellular compartment of the endothelial cells, which induce apoptosis or inhibit proliferation, or both.

Combining siRNA Oligos for Silencing Multiple Pathways

The processes leading to excessive and unwanted ocular neovascularization are complex and generally involve parallel biochemical pathways. As a result, therapeutic intervention at one target or even one pathway can be incomplete in control of disease pathology (neovascularization). The invention provides for combined intervention: intervention in multiple targets of a biochemical pathway or intervention in multiple pathways or both. For example, the invention provides for intervention with multiple targets of the VEGF pathway including a combination of siRNA for VEGF and VEGF-R2. The invention further provides for intervention in multiple pathways including the VEGF pathway targets. The invention also provides for combinations of these combinations, e.g. combined siRNA duplexes targeting both VEGF pathway and TGFβ pathway. This combination will have therapeutic benefit for both anti-angiogenesis and anti-scaring which is a common problem for patients with retina neovascularization condition, since the scar resulted from the anti-angiogenesis treatment will cause major vision impairment. Surgical removal of the scar from the retina tissue is very tedious and ineffective.

Delivery of Therapeutic Agents Through Local and Topical Applications

The invention provides compositions and methods for administering the therapeutic agents to treat ocular neovascularization diseases, in both anterior and posterior of the eye. The disease tissues anywhere in the eye can be treated with neovasculature-targeted delivery of therapeutic agents, according to the invention, by local administration, by topical administration to the eye, or by intravenous administration at a distal site. The disease tissues in the anterior of the eye can be treated, according to the invention, by local administration into the subconjunctival tissue, by topical administration to the eye, by periocular injection, by intraocular injection, and by intravenous administration at a distal site. The compositions provided by the invention include 1) cationic agents that bind nucleic acids by an electrostatic interaction, including non-natural synthetic polymers, grafted polymers, block copolymers, peptides, lipids and micelles, 2) hydrophilic agents that reduce non-specific binding to tissues and cells, including non-natural synthetic polymers, peptides, and carbohydrates, 3) tissue and cell penetrating agents, including surfactants, peptides, non-natural synthetic polymers, and carbohydrates, and 4) chemically modified siRNA oligos with Phosphorothioate, Boranophosphate, Methylphosphonate and Phosphodiester.

A preferred class of peptide is the histidine-lysine copolymer that is a basic, cationic, broad class of peptides, referred to in some instances as HKP. Another preferred class of peptide is linear polylysine with histidine or imidazole monomers coupled to the epsilon amino moiety of the lysine monomers. Another preferred class of peptide is branched polylysine and branched polylysine with histidine or imidazole monomers coupled to the epsilon amino moiety of the lysine monomers. A preferred composition has a self-assembled complex of negatively charged therapeutic agent such as a nucleic acid with a cationic peptide with an excess of cationic charge of 2 fold to 10 fold and a more preferred cationic charge of 2 fold to 6 fold. A preferred class of polylysine coupled with histidine or imidazole monomers has 30 to 70% coupling to primary amines of the lysine monomers. Another preferred class of peptide is a polymer with a monomer comprised of the tripeptide histidine-histidine-lysine or the tetrapeptide of histidine-histidine-lysine-lysine, where the polymer is either linear or branched, the branched polymer having monomers coupled to either the alpha or epsilon amino group of another monomer, or both. A preferred molecular weight of the polylysine class of polymers is in the range of 5,000 to 100,000, and a more preferred molecular weight of 10,000 to 30,000.

A preferred class of grafted polymers is a peptide grafted with a hydrophilic polymer, where the hydrophilic polymer includes PEG, polyoxazoline, polyacetal (referred to in some instances as Fleximer), HPMA, and polyglycerol. A preferred composition has a self-assembled complex of negatively charged therapeutic agent such as a nucleic acid with a cationic grafted polymer with an excess of cationic charge of 2 fold to 10 fold and a more preferred cationic charge of 2 fold to 6 fold. A preferred molecular weight of the hydrophilic polymer is in the range of 2,000 to 10,000. Another preferred class of grafted polymers is a peptide grafted with a hydrophilic polymer further comprised of a ligand grafted to the hydrophilic polymer, where the ligand includes peptides, carbohydrates, vitamins, nutrients, and antibodies or their fragments.

A preferred class of non-natural synthetic cationic polymer is a polymer with a backbone repeating unit of ethyl-nitrogen (—C—C—N—), including polyoxazoline and polyethyleneimine (PEI). A preferred composition has a self-assembled complex of negatively charged therapeutic agent such as a nucleic acid with a cationic polymer with an excess of cationic charge of 2 fold to 10 fold and a more preferred cationic charge of 2 fold to 6 fold. In one embodiment, the invention provides linear polyoxazoline or PEI derivatized with histidine or imidazole monomers. Another preferred class of polymer is branched polyoxazoline or PEI derivatized with histidine or imidazole monomers. A preferred class of polymer coupled with histidine or imidazole monomers has 30 to 70% of the basic moieties being imidazole. A preferred molecular weight of the polymers is in the range of 5,000 to 100,000, and a more preferred molecular weight of 10,000 to 30,000.

A preferred class of grafted polymers is a polymer grafted with a hydrophilic polymer, where the hydrophilic polymer includes PEG, polyoxazoline, polyacetal (referred to in some instances as Fleximer), HPMA, and polyglycerol. A preferred composition has a self-assembled complex of negatively charged therapeutic agent such as a nucleic acid with a cationic grafted polymer with an excess of cationic charge of 2 fold to 10 fold and a more preferred cationic charge of 2 fold to 6 fold. Another preferred class of grafted polymers is a polymer grafted with a hydrophilic polymer further comprised of a ligand grafted to the hydrophilic polymer, where the ligand includes peptides, carbohydrates, vitamins, nutrients, and antibodies or their fragments.

Another preferred class of cationic polymer is a polymer with a polyacetal backbone. A preferred composition has a self-assembled complex of negatively charged therapeutic agent such as a nucleic acid with a cationic polyacetal polymer with an excess of cationic charge of 2 fold to 10 fold and a more preferred cationic charge of 2 fold to 6 fold. In one embodiment, the invention provides linear polyacetal derivatized with a basic moiety, where the basic moiety class includes mixture of lysine, primary amine, histidine, and imidazole monomers. Another preferred class of polymer is branched polyacetal derivatized with a basic moiety (again including the class of lysine, amine, histidine, and imidazole monomers). A preferred class of polyacetal polymer coupled with lysine, amine, histidine, and imidazole monomers has 30 to 70% if the basic moieties being imidazole. A preferred molecular weight of the polymers is in the range of 5,000 to 100,000, and a more preferred molecular weight of 10,000 to 30,000. A preferred class of grafted polymers is a polymer grafted with a hydrophilic polymer, where the hydrophilic polymer includes PEG, polyoxazoline, polyacetal (referred to in some instances as Fleximer™), HPMA, and polyglycerol. Another preferred class of grafted polymers is a polyacetal polymer grafted with a hydrophilic polymer further comprised of a ligand grafted to the hydrophilic polymer, where the ligand includes peptides, carbohydrates, vitamins, nutrients, and antibodies or their fragments.

A preferred class of micelle is a block copolymer with one block comprised of a hydrophilic polymer and another block comprised of a hydrophobic polymer, including polypropylene oxide, a hydrophobic polyoxazoline, a hydrophobic polymer derivatized with primary amines or imidazole or both, a hydrophobic polymer dervatized with a moiety that forms a cleavable linkage with the therapeutic agent including a sulfydryl for a disulfide, an aldehyde for a Schiff's base, and an acid or alcohol for an ester. A preferred composition has a self-assembled complex of negatively charged therapeutic agent such as a nucleic acid with a micelle with an excess mass of micelle over that of the therapeutic agent of 2 fold to 50 fold and a more preferred excess mass of 4 fold to 20 fold. Another preferred class of micelle is a block copolymer further comprised of a ligand grafted to the hydrophilic polymer, where the ligand includes peptides, carbohydrates, vitamins, nutrients, and antibodies or their fragments.

The invention provides for formulated delivery of therapeutic agents including siRNA into cells and tissues. The formula provides protection of nucleic acids from degradation and facilitates the tissue and cellular uptake of the therapeutic agent. By delivering RNAi or other negatively charged therapeutic agents with a formulation composition of the invention, the invention achieves cellular uptake of the therapeutic agent and inhibition of expression of an endogenous target gene. By the use of a formulation for local administration, the invention provides for local administration of siRNA and other therapeutic agents into eyes for treatment of ocular disease, including intrastromal infections, corneal neovascularization, stromal keratitis, uveitis, etc. Although local administration may be more invasive than distant systemic delivery, and prone to risk of infection or irritation leading to inflammation, the local delivery of siRNA may still be preferred in many clinical situations, e.g., in severe NV conditions or in fast growing tumors. The invention also provides for incorporation of agents that increase the tissue adhesion and permeability through the corneal epithelium. This combination provides topical application in the form of eye-drops.

By use of the compositions and methods of the invention, siRNA and other therapeutic agents are used to treat ocular neovascularization by topical, local, or I.V. injection.

The following examples illustrate certain aspects of the invention and should not be construed as limiting the scope thereof.

### EXAMPLES

### Example 1

### 25 Mer siRNA is More Potent than 21 Mer siRNA

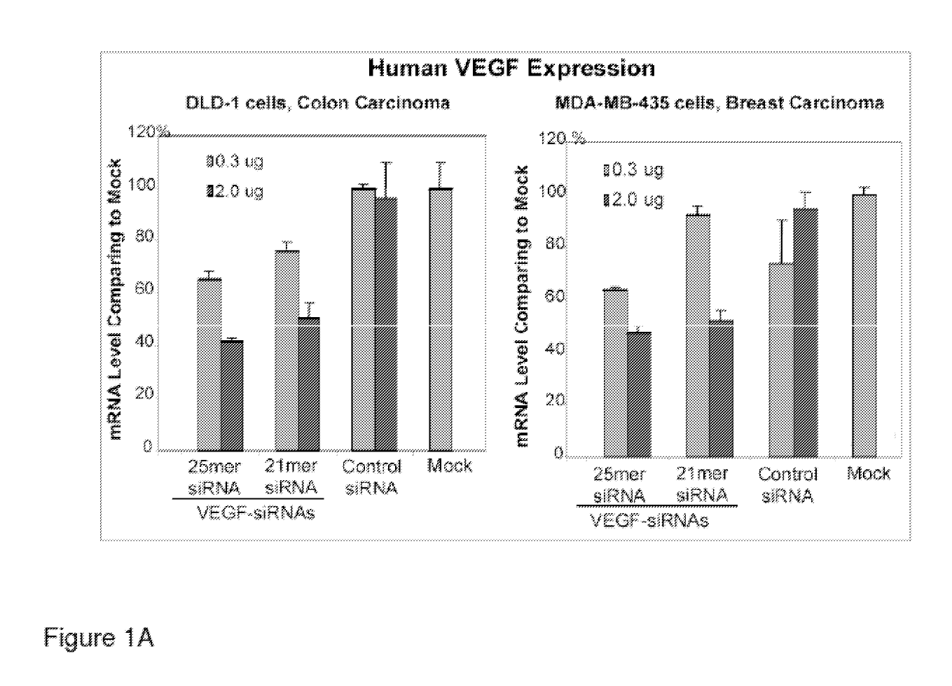

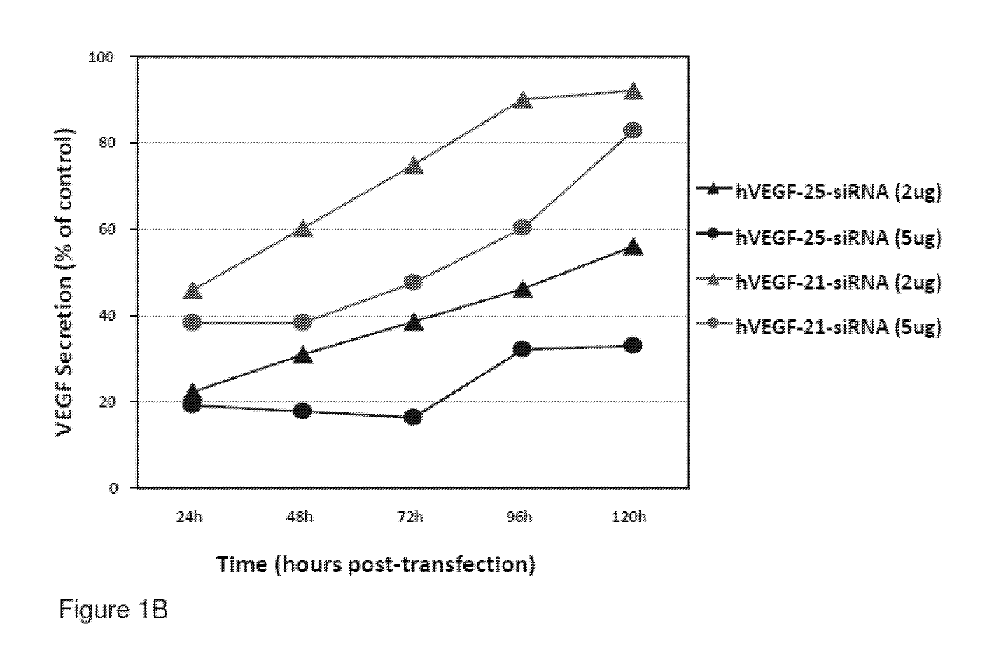

Although the initial studies were mostly utilizing 19mer and 21mer siRNA duplexes synthesized chemically, there is evidence showing that 23mer, 25mer and 27mer siRNA duplexes exhibited more potent silencing effects than the 19mer and 21mer siRNA oligos. The potential interferon pathway activation by longer siRNA oligos (23mer or longer) is a cell type dependent phenomenon. We found that 25mer duplexes with blunt ends are the most potent inhibitors, up to 60% either MBA-MD-435 or DLD-1 cells and in tumor bearing in animals. We have tested a 25mer siRNA duplex targeting human VEGF gene, hVEGF-25c (sense: 5′-CACAACAAAUGUGAAUGCAGACCAA-3′; Antisense: 5′-UUGGUCUGCAUUCACAUUUGUUGUG-3′), comparing to a 21mer siRNA duplex which has been tested many times as one of the most potent VEGF specific inhibitory duplexes, hVEGF-21a (sense: 5′-UCGAGACCCUGGUGGACAUTT-3′; antisense: 5′-AUGUCCACCAGGGUCUCGATT-3′), in the cell culture followed with Q-RT-PCR analysis (FIG. 1A). A similar study also carried out with ELISA analysis for the difference between the 25 mer and 21 mer siRNA both targeting VEGF (FIG. 1B).

### Example 2

### Selected siRNA is Specific to Both Human and Mouse VEGF mRNAs

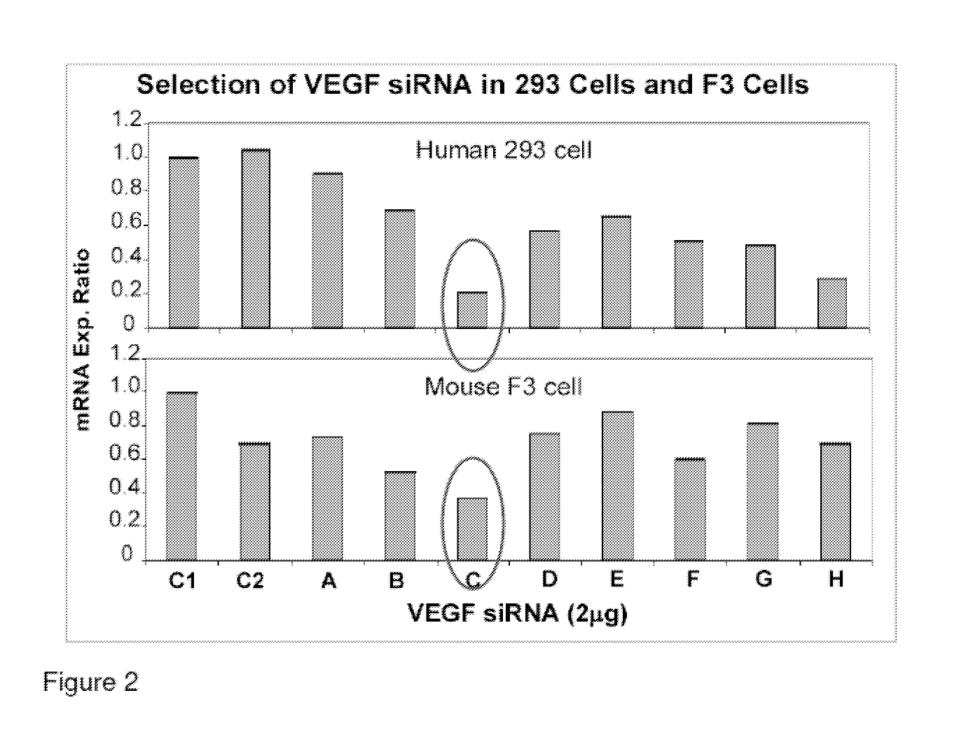

Using an in silico algorithm, we have designed eight siRNA duplex sequences (Table 1) for each gene target with following characteristics: a. optimum thermodynamics; b. enhanced RISC binding; c. eliminated immune stimulation motifs; d. having human and mouse homology; e. intellectual property searched; f. “Off Target” potential blasted and g. can be used as siRNA cocktail. The potent siRNA duplexes targeting each of the targets have followed by Q-RT-PCR (MyiQ, BioRad). The 25 mer siRNA duplexes were synthesized by Qiagen (Germantown, Md.) for in vitro cell culture studies, or by Dharmacon (Bolder, Colo.) at larger quantity for in vivo study with animal disease models. The cell lines used in the studies for potent siRNA selections are due to the target gene expressions in those cells. For example, both human 293 cells and mouse F3 cells were used for selection of VEGF specific siRNA duplex (FIG. 2). The most potent siRNA duplex targeting VEGF, hmVEGFc: 5′-CUGUAGACACACCCACCCACAUACA-3′ (sense), was selected as the active pharmaceutical ingredient (API) for VEGF gene silencing.

### Example 3

### Selected siRNA is specific to both human and mouse VEGFR2 mRNAs

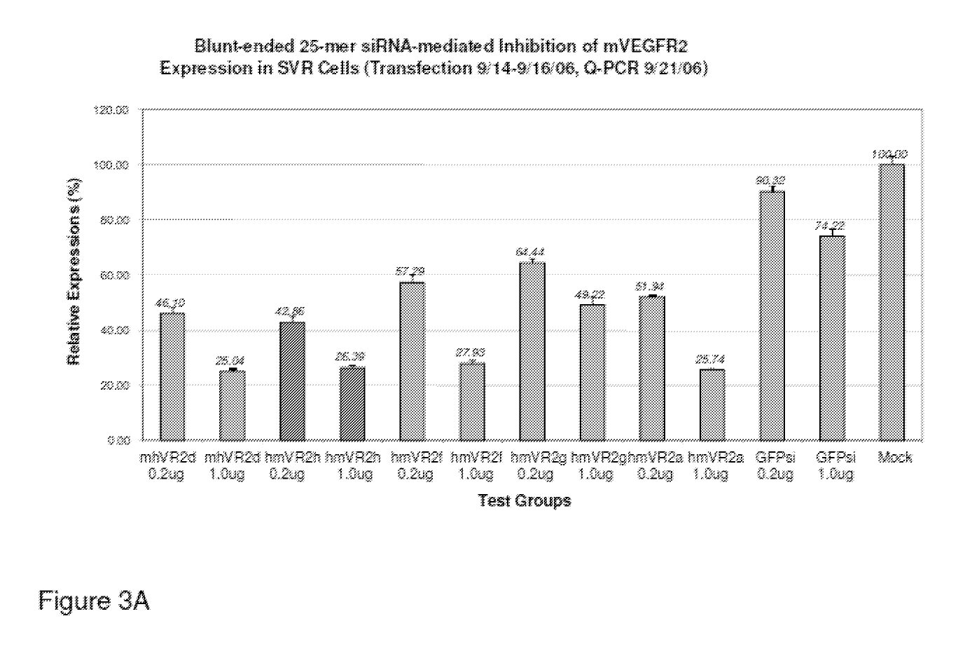

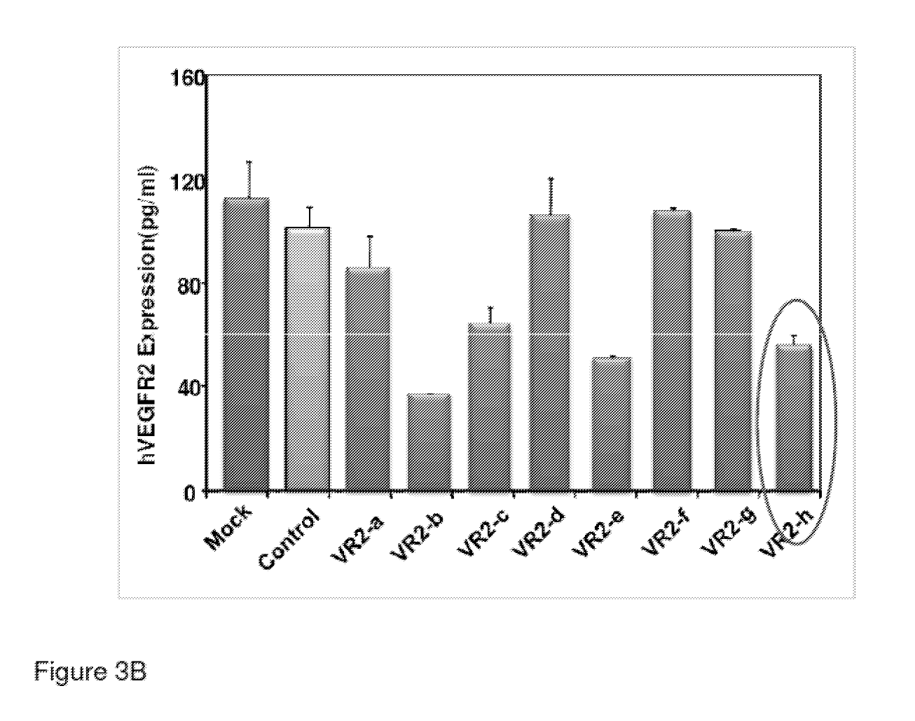

Using the in silico algorithm, we have designed eight siRNA duplex sequences (Table 2) for each gene targets with following characteristics: a. optimum thermodynamics; b. enhanced RISC binding; c. eliminated immune stimulation motifs; d. having human and mouse homology; e. intellectual property searched; f. “Off Target” potential blasted and g. can be used as siRNA cocktail. The 25 mer siRNA duplexes were synthesized by Qiagen (Germantown, Md.) for in vitro cell culture studies, or by Dharmacon (Bolder, Colo.) at larger quantity for in vivo study with animal disease models. The cell lines used in the studies for potent siRNA selections are due to the target gene expressions in those cells. The mouse SVR cells were transfected with siRNA duplexes followed by RNA isolation and Q-RT-PCR (FIG. 3A). The human HUVEC cells were transfected with siRNA followed by protein isolation and ELISA (FIG. 3B). These two assays were used for selection of VEGFR2 specific siRNA duplex. The most potent VEGFR2 siRNA, hmVR2h: 5′-GACUUCCUGACCUUGGAGCAUCUCA-3′ (sense), was select as the active pharmaceutical ingredient for silencing VEGFR2 gene expression.

### Example 4

### Selected siRNA is specific to both human and mouse TGFβ1 mRNAs

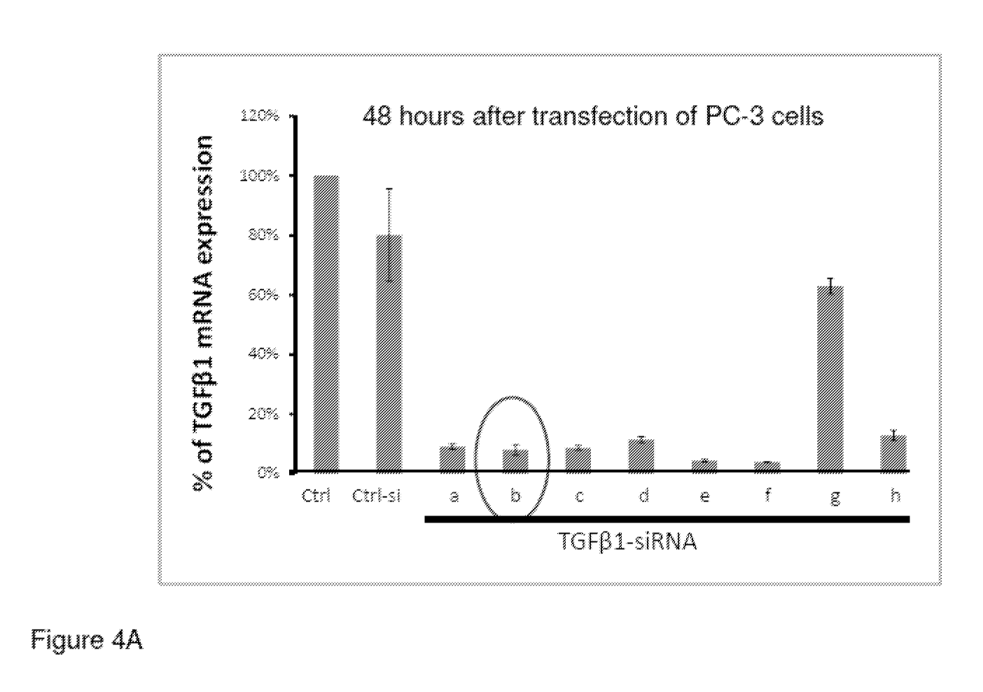

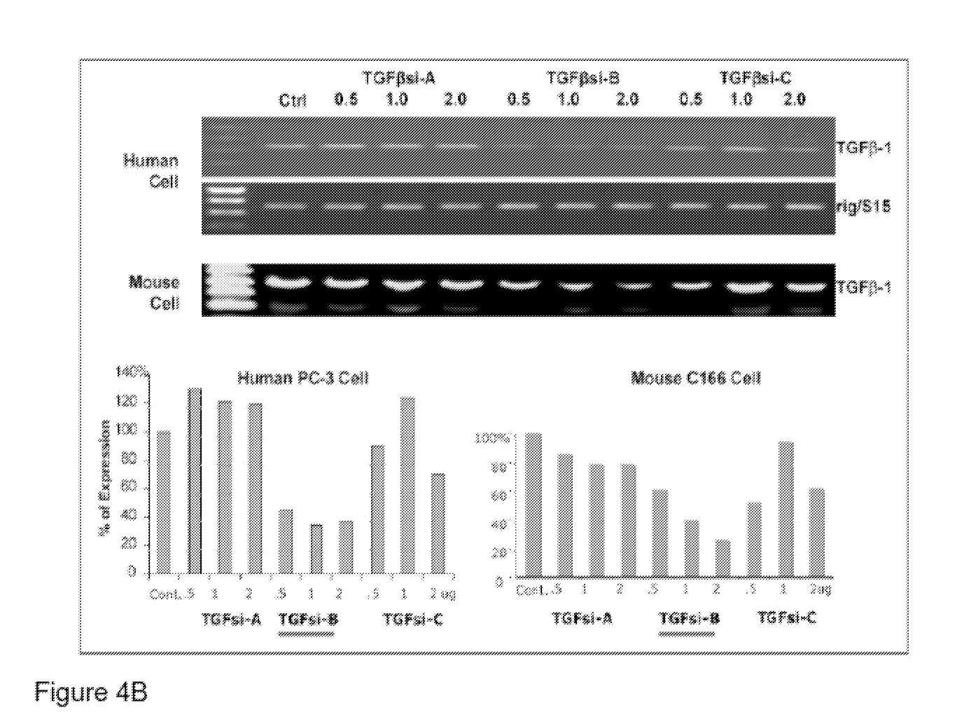

Using the in silico algorithm, we have designed eight siRNA duplex sequences (Table 3) for each gene targets with following characteristics: a. optimum thermodynamics; b. enhanced RISC binding; c. eliminated immune stimulation motifs; d. having human and mouse homology; e. intellectual property searched; f. “Off Target” potential blasted and g. can be used as siRNA cocktail. The 25 mer siRNA duplexes were synthesized by Qiagen (Germantown, Md.) for in vitro cell culture studies, or by Dharmacon (Bolder, Colo.) at larger quantity for in vivo study with animal disease models. The potent siRNA duplexes targeting each of the targets have followed by Q-RT-PCR (MyiQ, BioRad). The cell lines used in the studies for potent siRNA selections are due to the target gene expressions in those cells. For example, both human PC3 cells (FIG. 4A-B) and mouse C166 cells (FIG. 4B) were used for selection of TGFβ1 specific siRNA duplex. The most potent siRNA targeting TGFβ1, hmTF25f: 5′-GAGGUCACCCGCGUGCUAAUGGUGG-3′(sense), was selected as the active pharmaceutical ingredient for silencing TGFβ1 gene expression.

### Example 5

### Selection of siRNA Combination as the Drug Candidates

To improve the potency of siRNA therapeutics and fully take advantages of this novel drug modality using combination of two siRNA duplexes or three siRNA duplexes, we have made following combinations:

(1) Combination 1, VEGF-VEGFR2 siRNA duplexes:
VEGF specific siRNA hmVEGFc: 5′-CUGUAGACACACCCACCCACAUACA-3′ (sense) is combined with hmVR2h: 5′-GACUUCCUGACCUUGGAGCAUCUCA-3′ (sense) as a dual target siRNA therapeutic API.
   (2) Combination 2, VEGF-TGFβ1 siRNA duplexes:
VEGF specific siRNA hmVEGFc: 5′-CUGUAGACACACCCACCCACAUACA-3′ (sense) is combined with hmTF25f: 5′-GAGGUCACCCGCGUGCUAAUGGUGG-3′(sense) as a dual target siRNA therapeutic API.

### (3) Combination 3, VEGF-VEGFR2-TGFβ1:

VEGF specific siRNA hmVEGFc: 5′-CUGUAGACACACCCACCCACAUACA-3′ (sense) is combined with hmVR2h: 5′-GACUUCCUGACCUUGGAGCAUCUCA-3′ and further combined with hmTF25f: 5′-GAGGUCACCCGCGUGCUAAUGGUGG-3′(sense) as a triple target siRNA therapeutic API.

### Example 6

### HKP-siRNA is able to self assemble into nanoparticle

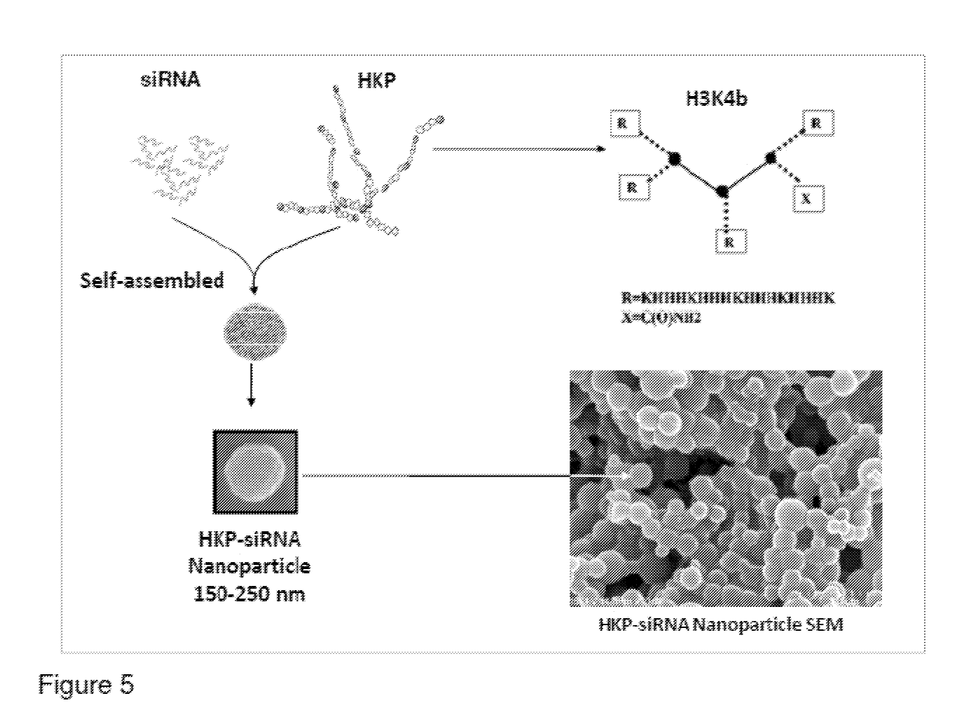

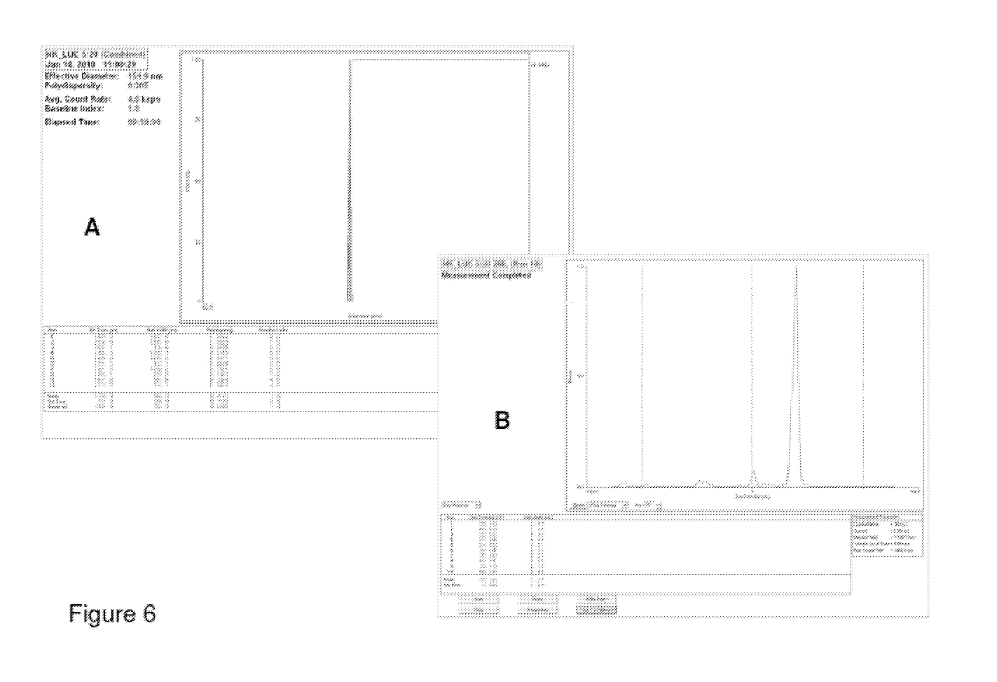

Optimized branched histidine-lysine polymers (HKP) have been applied for siRNA deliveries in vitro and in vivo. A pair of the HK polymer species, H3K4b and H3K_{(+H)}4b, has a Lysine backbone with four branches containing multiple repeats of Histidine, Lysine or Asparagine. When this HKP aqueous solution was mixed with siRNA at a N/P ratio of 4:1 by mass, the nanoparticles (average size of 100-200 nm in diameter) were self-assembled (FIG. 3). Optimal branched histidine-lysine polymer, HKP, was synthesized on a Ranin Voyager synthesizer (PTI, Tucson, Ariz.). The two species of the HKP used in the study were H3K4b and H3K_{(+H)}4b with a structure of (R)K(R)-K(R)-(R)K(X). For H3K4b where R=KHHHKHHHKHHHKHHHK; and for H3K_{(+H)}4b R=[KHHHKHHHHKHH-HKHHH], X═C(O)NH2, K=lysine, H=histidine and N=Asperagine. The HKP was dissolved in aqueous solution and then mixed with siRNA aqueous solution at a ratio of 4:1 by mass, forming nanoparticles of average size of 150-200 nm in diameter (FIG. 5). The HKP-siRNA aqueous solution was semi-transparent without noticeable aggregation of precipitate, and can be stored at 4° C. for at least three months. The characterization of HKP-siRNA nanoparticle has been described with particle size and Zeta potential (FIG. 6). We applied both H3K4b and H3K_{(+H)}4b for delivery of siRNA into ocular and other tissue types with high efficiency.

### Example 7

### HKP-siRNA is Able to Enhance Local siRNA Delivery into Mouse Eye

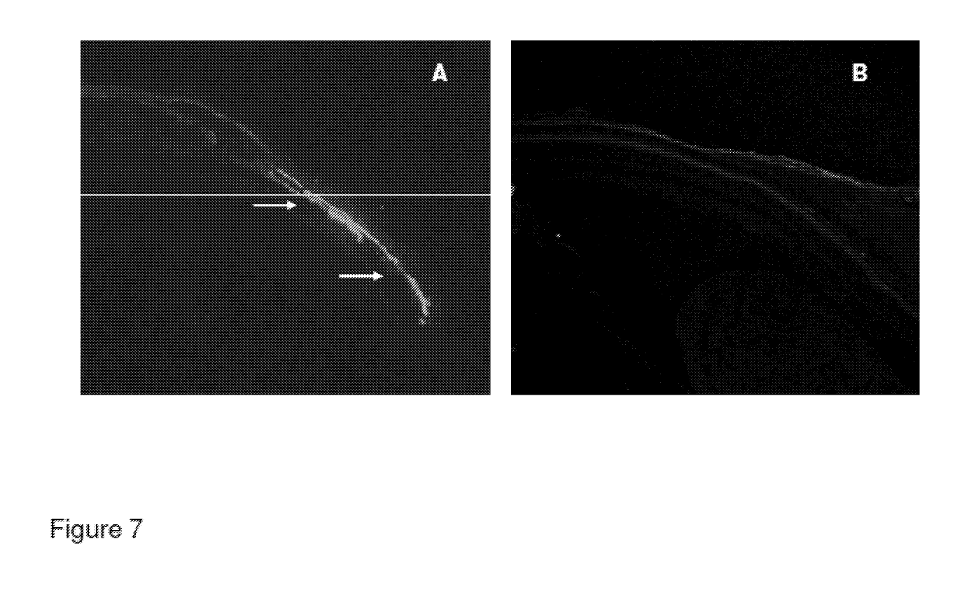

The mouse Herpetic Stromal Keratitis was generated with CpG-ON pellet implant, which represents the clinically relevant models of corneal neovascularization with typical characteristics of inflammation-induced angiogenesis and lymphangiogenesis. To evaluate the efficacy of siRNA delivery with HKP nanoparticle using CpG pellet induced corneal inflammation, the FITC-labeled siRNA was delivered through subconjunctival injection and observed in angiogenic corneal cryosection 24 hr after the administration. The comparison indicated that the labeled siRNA is accumulated in the corneal tissue from the cryosection of the group treated with HKP-FITC-labeled siRNA, versus very weak signal was observed even through the same route of delivery (FIG. 7). This result is the direct evidence that HKP is able to facilitate corneal siRNA delivery through subconjunctival administration. The HKP-siRNA demonstrated a control release pattern with large amount of siRNA preserved around the injection site. This phenomenon has been observed with other tissue types when the same HKP-siRNA nanoparticle was delivered.

### Example 8

### siRNA Cocktail Exhibits Potent Anti-Angiogenesis Activity

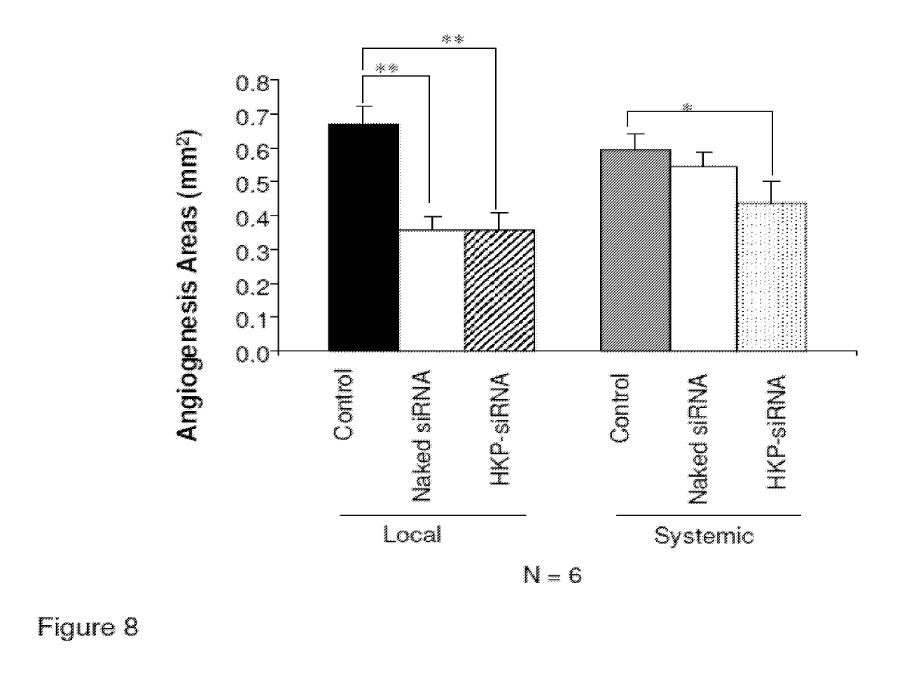

The mouse Herpetic Stromal Keratitis was generated with CpG-ON pellet implant and HSV infection as described previously, which represents the clinically relevant models of corneal neovascularization with typical characteristics of inflammation-induced angiogenesis and lymphangiogenesis. Measurements of the angiogenesis areas and HSK disease scores are the most effective ways to evaluate the siRNA-mediated anti-angiogenesis activities in the anterior section of the eyes. We discovered that knocking down VEGF, VEGFR1 and VEGFR2 individually resulted in similar anti-angiogenesis effects in these corneal angiogenesis models and combining multiple siRNA duplexes targeting all three genes resulted in stronger anti-angiogenesis activity. In this study, we found that HKP-siRNA cocktail resulted in more potent anti-angiogenesis activities than the naked siRNA cocktail (FIG. 8) in the CpG induced angiogenesis model.

### Example 9

### HKP-siRNA Formulation for Intravitreal Delivery in Rabbit Eyes

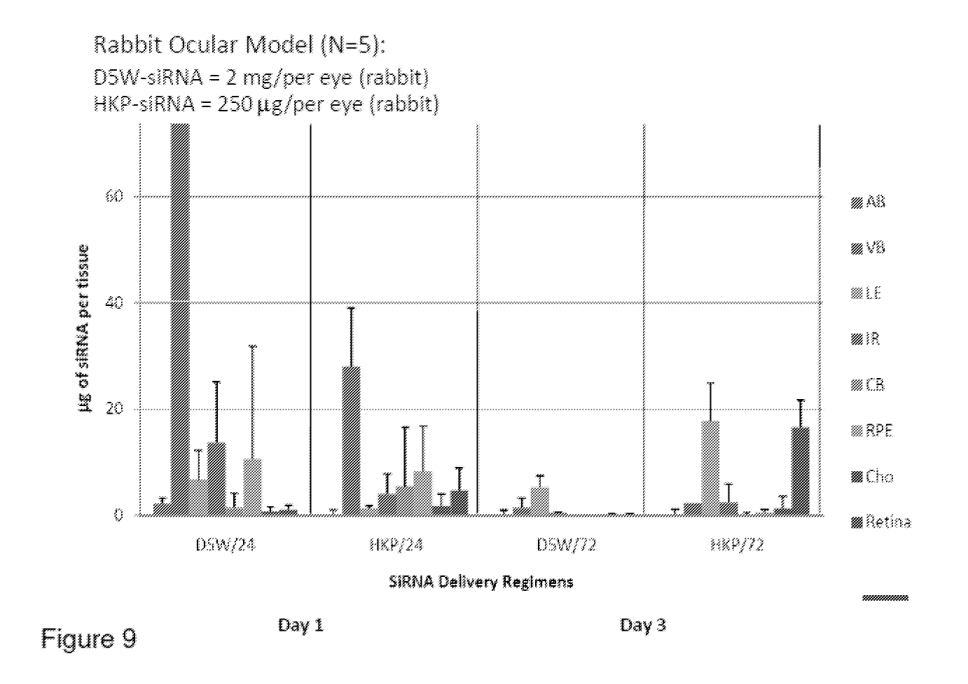

3H-labeled siRNA was used for dosing rabbit eyes (female Dutch-Belted rabbits) were used. 0.5 mg 3H-labeled siRNA was used with a dose volume of 50 μl/eye, using a binocular indirect ophthalmoscope to confirm needle placement. The ophthalmologist examined the eyes immediately following treatment (indirect ophthalmoscopy and slit-lamp biomicroscopy) and documented any abnormalities caused by the dosing procedure. Following examination, gentamycin ophthalmic drops were applied to each eye and an ocular lubricant (Tears Naturale®, Alcon, Fort Worth, Tex.) was used if considered appropriate by the ophthalmologist. Animals were euthanized at predetermined times (24 and 72 h post-injection) by intravenous injection of Euthanyl® (Bimeda-MTC Animal Health Inc., Cambridge, Ontario, Canada; approximately 200 mg/kg). Four animals were sacrificed at each time point. Following euthanasia, tissue was collected. The right eye was removed intact. The left eye was dissected to collect aqueous fluid, iris, vitreous fluid, retina and sclera (including choroid). All samples were stored at −80° C. Radioactivity measurements: The total weights of the tissue samples were recorded. Tissue samples were solubilized in 35% tetraethylammonium hydroxide (TEAH). The solubilized samples, or duplicate aliquots thereof, were then mixed with liquid scintillation fluid before radioactivity measurements. Radioactivity measurements were conducted by liquid scintillation spectroscopy. Each sample was counted for 5 min or to a two-sigma error of 0.1%, whichever occurred first. All counts were converted to absolute radioactivity (dpm) by automatic quench correction based on the shift of the spectrum for the external standard. Samples that exhibited radioactivity less than or equal to twice the background values were considered as zero for all subsequent manipulations. All radioactivity measurements were entered into a standard computer database program (Debra Version 5.2) for the calculation of concentrations of radioactivity (dpm/g and mass eq/g) and percentage of administered radioactivity in each sample. Tissue concentrations of radioactivity were calculated initially in dpm/g, and then mass eq/g (assuming intact siRNA) was calculated on the basis of the measured specific activity (dpm/mg or appropriate mass unit) of radiolabeled test article in the dose solution. Total tissue content was calculated based on the total tissue weight. Non-compartmental pharmacokinetic parameters were estimated for the ocular tissue data, using SAS Version 8.1, and included area under the concentration versus time curve (AUC), terminal half-life (t½el), terminal rate constant (kel), the highest concentration observed (Cmax), and time at which the highest concentration occurred (tmax). The Cmax was obtained by data inspection. The AUC was calculated by application of the trapezoidal rule and kel was obtained by linear regression analysis of selected time points in the terminal phase of the concentration versus time curves. The apparent terminal half-life (t½el) was calculated as follows: t½el=ln2/kel. For all time deviations greater than 10%, the actual time collection was used for estimation of the parameters. The results are illustrated in FIG. 9 showing the radioactivity of each tissue type at 24 and 72 hours post siRNA administration. Clearly, even though the initial injection of naked siRNA is more (2 mg/per eye) than the HKP-siRNA (250 μg/per eye), the total siRNA recovered at 72 hours post administration is much higher for HKP-siRNA than those from naked siRNA (FIG. 9).

### Example 10

### HKP-siRNA Nanoparticle Enhances Retina siRNA Delivery

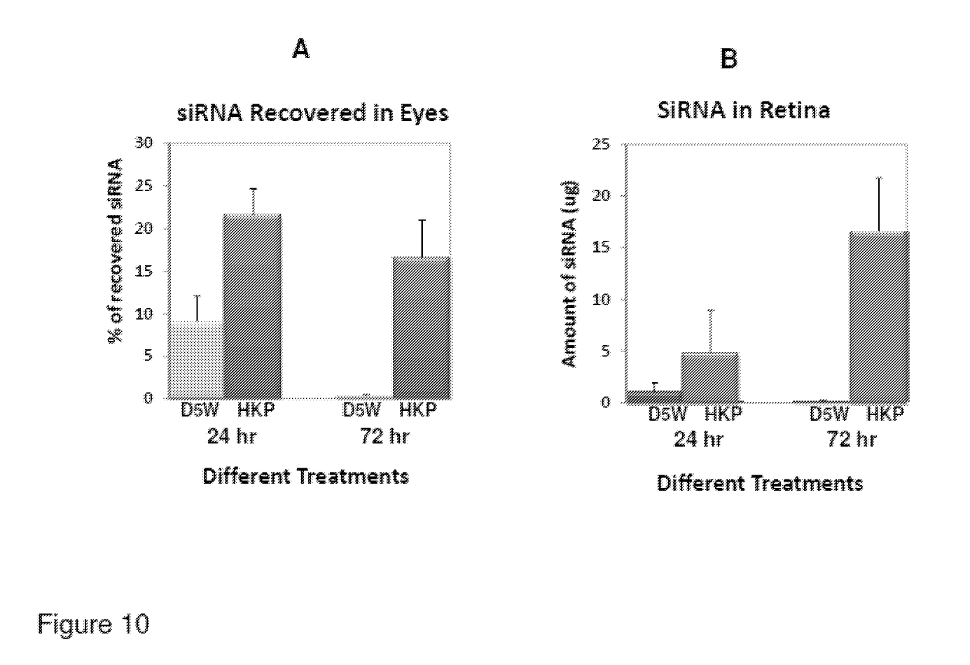

From the samples of 3H-labeled rabbit ocular tissues, we found that HKP-siRNA nanoparticle is much more stable than those of naked siRNA. Not only the total rate of recovery is much higher for HKP-siRNA than those from the naked siRNA, but the absolute number of 3H-labeled HKP-siRNA is also much higher in the retina tissue than those of 3H-labeled naked siRNA. This result shows that the HKP-siRNA injected by intravitreous administration is able to stabilize siRNA and accumulate siRNA into the retina tissue (FIG. 10). The tissue distributions of siRNA within the eye are quite different at day one and day three: majority of the injected siRNA is located in the vitreous body at day 1 time point but shifted to lens and retina at day 3. This shift and accumulation to the lens and retina are very prominent when the siRNA is packaged with HKP. Therefore, we have speculated that HKP-siRNA nanoparticle is able to targeted to the retina tissue through a unknown mechanism of action which will be very beneficial to such a treatment.

### Example 11

### Therapeutic Benefit Observed Through Hypoxia Induced Retinopathy

Hypoxia induced retinopathy (HIR) model was established with C57BL/6 mice purchased from Center of Experimental animal of Guangzhou Medical college, Guangzhou University of traditional Chinese Medicine. Briefly, the pups with the nursing dams were maintained in hyperoxia environment (75%+2 oxygen) from postnatal days P7 to P12, then returned to room air (normoxia), followed by treatment with Polymeric siRNA nanoparticles via different routes of delivery. The ocular NV in OIR model was evaluated with fluorescein perfusion/flatmounting, cryosection staining, RT-PCR for mRNA levels and ELISA for protein levels. All investigations followed guidelines of the Committee on the Care of Laboratory Animals Resources, Commission of Life Science, National Research Council, China.

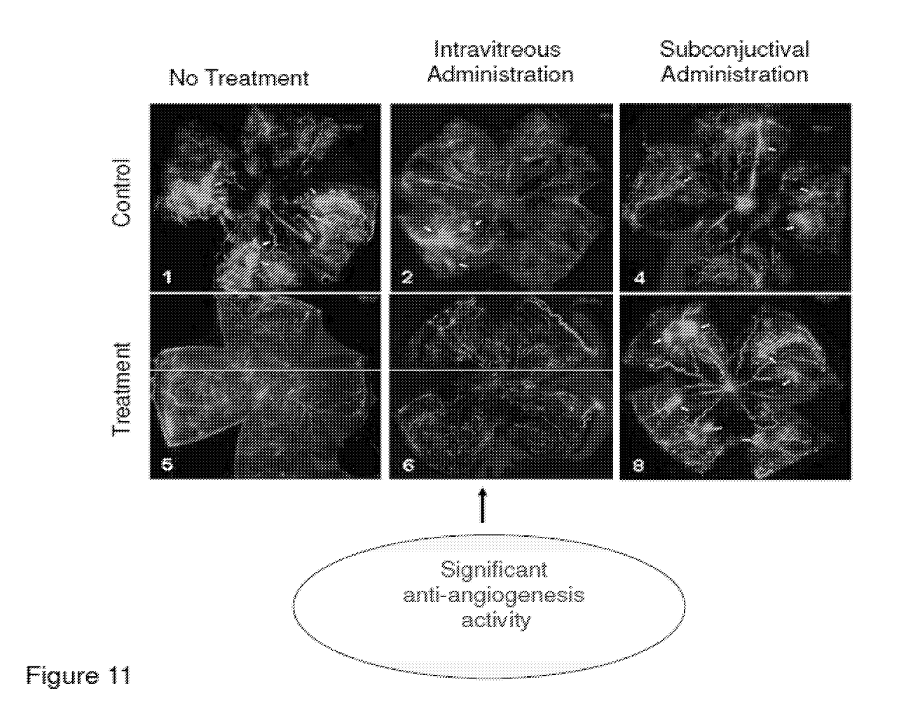

Since hypoxia induced angiogenesis model reflects a choroidal neovascularisation (CNV) with pathogenesis of ischemic and degenerative diseases, we tested the HKP-siRNA cocktail nanoparticle formulation through both local (intravitreal) and systemic (intraperitoneal) administrations in such a model, to reveal its therapeutic potentials for treatments of retinopathy of prematurity and age-related macular degeneration. Two routes for local deliveries, subconjunctival and intravitreal, were applied for HKP-siRNA cocktail formulation, while the systemic delivery was switched from intravenous to intraperitoneal due to the size limitation of tail vein of the young mouse pubs. Administrations through all three routes were arranged into regimen A: injected twice at P12 and P13, and regimen B: injected three times at P12, P14 and P16, respectively. The samples were collected at P14 and P17, following cardiac perfusion with fluorescein-labeled dextran. Comparison of the retinal Flatmounts indicated that both intravitreous and intraperitoneal administrations worked well with either regimen A or B, achieving reduced angiogenesis areas about 50% (FIG. 11).

### Example 12

### Therapeutic Benefit Observed Via Cryosection of Samples from HIR Model

Eyes were enucleated and frozen in optimal cutting temperature embedding compound (Miles Diagnostics, PA, USA) for Cryosection analysis. Ocular frozen sections (10 μm) were histochemically stained with biotinylated GSA. Slides were incubated in methanol/H₂O₂ for 10 min at 4° C., washed with 0.05 M Tris-buffered saline (TBS), pH 7.6, and incubated for 30 min in 10% normal bovine serum. Slides were incubated with biotinylated GSA, avidin coupled to alkaline phosphatase (Vector Laboratories) and diaminobenzidine, further counterstained with eosin, and mounted with Cytoseal. To perform quantitative assessments, 15 GSA-stained sections were examined with microscope, and images were digitized using digital camera. Image-Pro Plus software (Media Cybernetics, Silver Spring, Md., USA) was used to delineate GSA-stained cells on the surface of the retina and measure the areas. In case of the local delivery, the measurement from each eye was used as a single experimental value. As for systemic delivery, the mean of both eyes of a mouse was considered as a single experimental value.

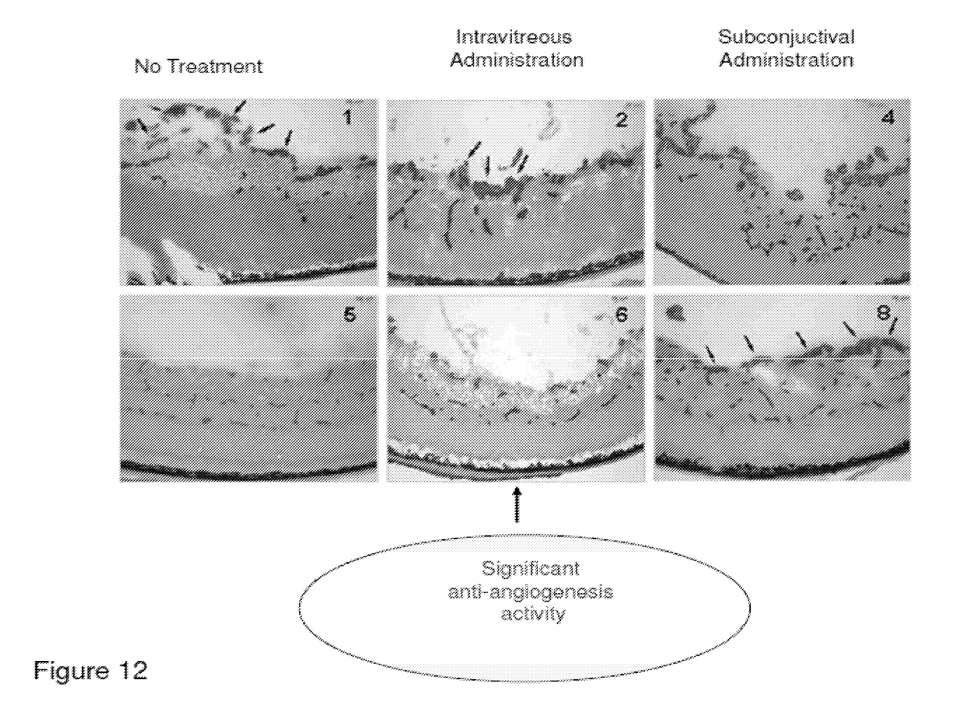

Using the cryosection analysis, retinal NV was assessed histologically by measured the GSA-positive cells anterior to the internal limiting membrane (ILM). The samples from the Intravitreous and intraperitoneal administration provided significantly reduced angiogenesis effect with both delivery regimens (FIG. 12). This interesting observation on the other hand confirmed that the intraperitoneal administration of HKP-siRNA cocktail was indeed reaching retina tissue mainly through the blood stream, while the subconjunctival delivered HKP-siRNA cocktail was unable to efficiently pass through blood-retinal barrier (BRB) and failed to reach CNV tissue.

### Example 13

### Proof of RNAi Mechanism of Action at mRNA Level

There is a report that a sequence- and target-independent angiogenesis suppression by siRNA via TLR3 was observed. However, we did not see such phenorminon when the nanoparticle enhanced siRNA delivery was carried out in the mouse ocular angiogenesis models, through detection of mRNA levels in cell culture and ocular tissues by RS-PCR and RT-PCR. Total RNA from transfected cells was extracted by RNAwiz (Ambion, #9736) and total RNA from retina was extracted using TRIzol reagent (Invitrogen, USA) after mice was sacrificed at P14 and P17. The cytoplasmic RNA samples were tested by mRNA-specific PCR (RS-PCR) as described previously ( ). The set of primers for each mRNA include a 47-mer mRNA-specific primer for reverse transcription reaction (RTP), a 5′-end gene specific primer (GP) and a 3′-end universal primer of 5′-GAACATCGATGACAAGCTTAGGTAT CGATA-3′. The primers for amplification of each gene are as follows: mVEGF: (RTP) 5′-GAACATCGATGACAAGCTTAGGTATCGATAcaagctgcctcgccttg-3′, (GP) 5′-GATGTCTA CCAGCGAAGCTACTGCCGTCCG-3′; and mVEGFR2 (RTP) 5′-GAACATCGATGACAAGCTTAGGTATCGATaggtcactgacagaggcg-3′, (GP) 5′-GGCGCTGCTAGCTGT CGCTCTGTG GTTCTG-3′. The lower cases indicated the sequences specific to the targets for reverse transcriptions. The RNA samples were also quantified with GAPDH and β-actin specific RT-PCR. All PCR products were subjected to the gel electrophoresis analysis and quantification.

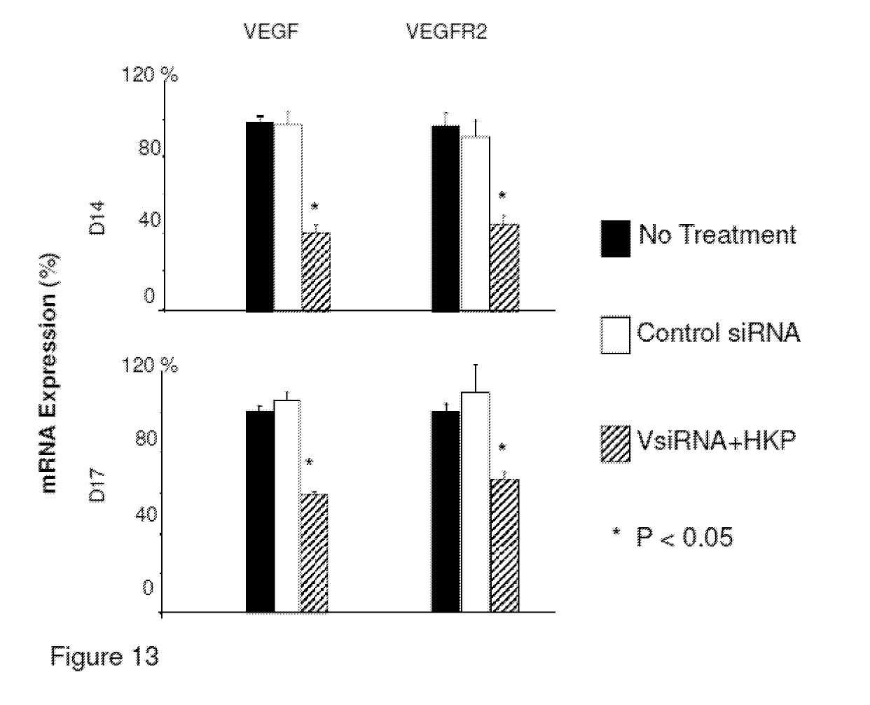

Seeing that the HKP-siRNA cocktail formulations clearly demonstrated the potent anti-angiogenesis efficacy in those mouse ocular neovascularization models, delivered through either local or systemic routes, we asked ourselves if it could be stipulated that these anti-angiogenesis benefits are indeed results of the siRNA-mediated gene silencing. The first evidence that we were looking for was the sequence- and target-dependent gene silencing at either mRNA. Analyzing mRNA samples from the eye tissues of HIR mouse model using Q-RT-PCR, we found strong knockdown of target gene expressions at mRNA of VEGF and VEGFR2 (FIG. 13), with a sequence- and target-dependent manner, regardless local or systemic administration. One thing we found was interesting, that VEGF expression was remarkably higher in the HIR eyes, but not for the VEGFR2 expression. Moreover, the endogenous VEGFR2 expression was able to be silenced by the nanoparticle-siRNA cocktail formulation at mRNA.

### Example 14

### Proof of RNAi Mechanism of Action at Protein Level

We also used ELISA analyses for VEGF and VEGFR2 knockdown at protein level. Retina were collected after mice were sacrificed at P14 and P17, and homogenized in cell lysis buffer (Mammalian cell lysis Kit, Biotechnology Department Bio Basid Inc, Canada). The supernatants were subjected to ELISA analysis using BCA protein quantitative analysis Kit (Shenery Biocolor Bioscience & Technolgy Company, China). Levels of VEGF and VEGFR2 were determined using the Quantikine M Murine VEGF and sVEGFR2 Immunoassay Kits respectively (R&D Systems Inc., Minneapolis, Minn.). Six to 12 tissue samples were analyzed for each group and each time point.

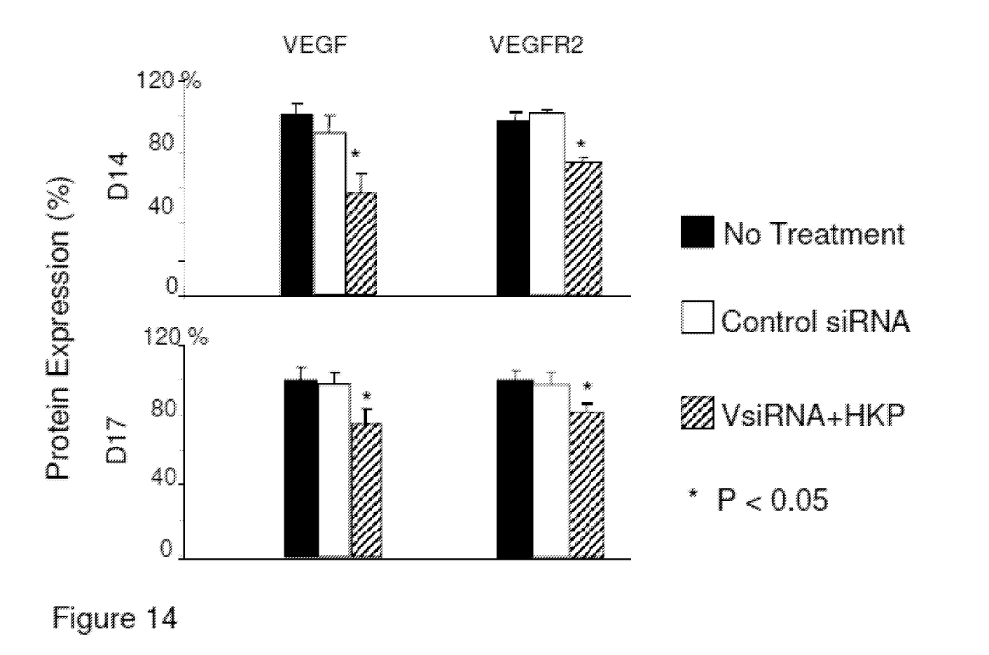

Seeing that the nanoparticle-simVmix formulations clearly demonstrated the potent anti-angiogenesis efficacy in those mouse ocular neovascularization models, delivered through either local or systemic routes, we wanted to make sure that these anti-angiogenesis benefits are indeed results of the siRNA-mediated gene silencing. In addition to the evaluation at the mRNA level, we also looked for the sequence- and target-dependent gene silencing at protein level. Analyzing protein samples from the eye tissues of HIR mouse model using ELISA, we found strong knockdown of target gene expressions at protein levels, with a sequence- and target-dependent manner, regardless local or systemic administration (FIG. 14). The siRNA cocktail packaged with HKP was effectively silencing VEGF and VEGFR2 expressions, with two different dose regimens and two sampling time points, day 14 and 17. The sequence- and target-dependent gene silencing was observed in the CpG induced and Herpes Virus induced mouse neovascularization models. Throughout our studies, targeting either single gene or multiple genes, using either local or systemic deliveries, and working with either retina or corneal neovascularization models, we have not seen sequence-independent anti-angiogenesis reported by others¹¹.

### Example 15

### Scarless Wound Healing with TGFβ1 siRNA in a Mouse Model

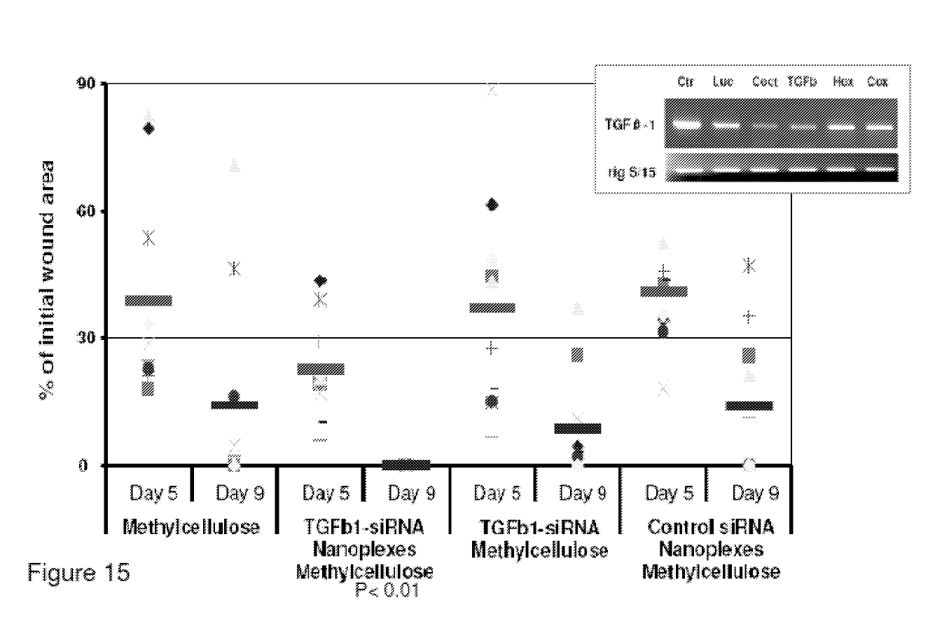

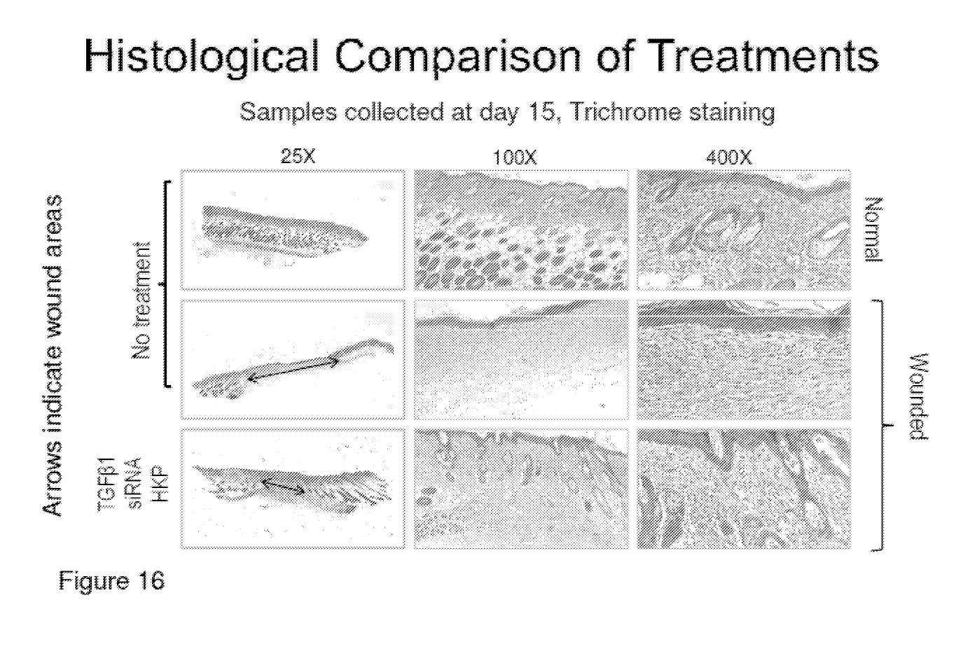

One major shortcoming after anti-angiogenesis treatment using an antagonist drug such as Avastin or Lucentis is scar formation on the retinal tissue, which severely hinders the therapeutic benefit of those medications and results in impaired visual acuity. One concept we developed is to use TGFβ1 siRNA for silencing this pro-inflammatory factor, in order to minimize the scar formation during the healing process after the anti-angiogenesis treatment. We have evidence that TGFβ1 siRNA is effective for enhancing wound closure and reducing scar formation when it was applied with a mouse skin excision wound model. When we used HKP-siRNA targeting TGFβ1 with a methylcellulose dressing on the skin excision wounds, a significantly improved speedy wound closure was observed compared to those treated with the control siRNA sequence packaged in the HKP and with HKP itself (FIG. 15). This observation was further supported by the histological analysis using the skin samples from the treated and untreated mice, with Trichrome staining (FIG. 16). The texture of the HKP-TGFβ1 siRNA treated skin wound tissue is very much like the normal skin texture comparing to the untreated mouse skin. Due to the literatures and the above evidence, we would like to propose that use the combination of siRNA duplexes targeting both angiogenesis pathway such as VEGF and VEGFR2, and TGFβ1 pathway, will represent a novel approach for treatment of ocular neovascularization diseases. We expect that such a treatment, with VEGF-TGFβ1 siRNA combination package in the HKP: VEGF specific siRNA hmVEGFc: 5′-CUGUAGACACACCCACCCACAUACA-3′ (sense) is combined with hmTF25f: 5′-GAGGUCACCCGCGUGCUAAUGGUGG-3′(sense) as a dual target siRNA therapeutic API, will not only reduce the neovasculature development but also minimize the scar formation, consequently, the vision acuity of the patient will be protected and improved.

### Example 16

### First Generation of Nanoparticles for Ocular siRNA Therapeutics

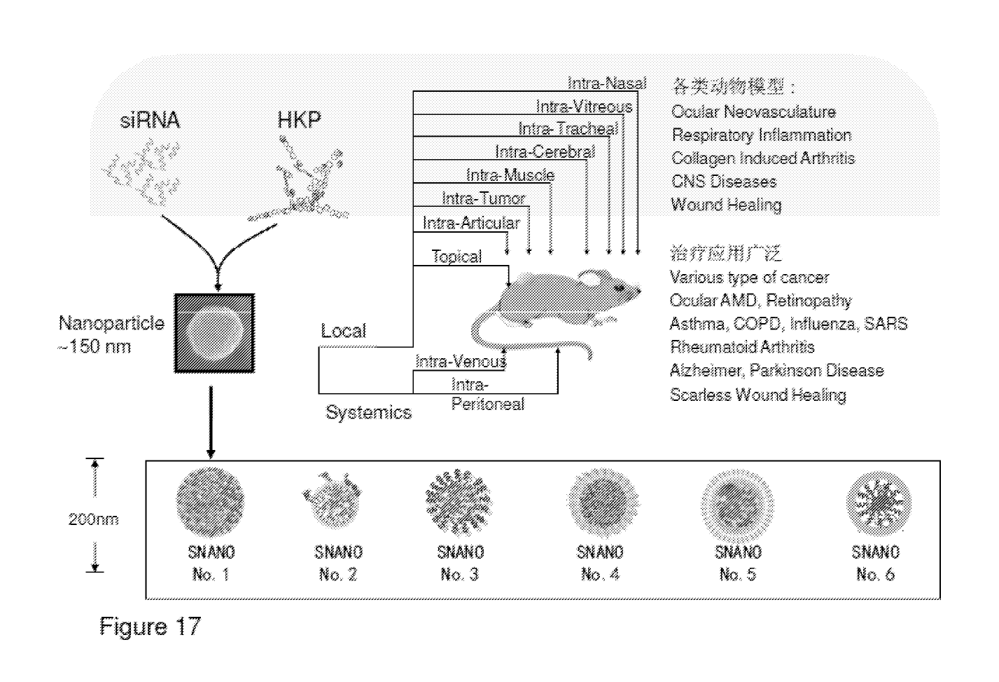

There are many different types of peptides, polymers, liposomes and other materials could be used as siRNA delivery vehicles for the therapeutic development. We suggested that the nanoparticle based on self assembling property of siRNA and cationic polymer or liposome through electrostatic interaction can be defined as the first generation of siRNA delivery carrier (FIG. 17).

HKP, indicated as Snano-1, is a typical first generation of siRNA delivery vehicle. The positively charged and branched histidine-lysine peptides and negatively charged siRNA are able to form a very homogenous nanoparticle population which is very effective for in vivo siRNA delivery.

Polyamidoamine dendrimer (PAMAM), indicated as Snano-2, is well-characterized, highly branched synthetic macromolecules that are biocompatible and nonimmunogenic, providing a unique platform for delivery of a variety of therapeutic agents, imaging agents, and oligonucleotides such as siRNA. A G5 PAMAM dendrimer is particular useful in our hand for siRNA delivery in vitro and in vivo.

Poly(lactic acid) (PLA), Poly(glycolic acid) (PGA), and their copolymers (PLGA), indicated as Snano-3, have been extensively investigated because of their biocompatibility and biodegradability. This material has been applied as potential carries for several classes of drugs such as anticancer agents, antihypertensive agents, immunomodulators, and hormones; and macromolecules such as nucleic acids, proteins, peptides, and antibodies. The options available for preparation have increased with advances in traditional methods, and many novel techniques for preparation of drug-loaded nanoparticles are being developed and refined. We have developed a method to prepare PLGA-siRNA nanoparticle into a smaller size about 200 nm in diameter which is very efficient for in vivo siRNA delivery.

Polyethylene glycol-polyethylenimine (PEG-PEI), nanoparticles, indicated as Snano-4, have been used to deliver nucleic acids and oligonucleotides in vivo. The small molecular weight of PEI can preserve the positive charge which is critical for forming a nanoparticle with negative charged siRNA, while reduce the toxicity due to excessive positive charge causing aggregation and non-specific binding after in vivo delivery. Pegylation can further improve the circulation time of the PEG-PEI nanoparticle within the blood stream.

### DOTAP

The use of DOTAP enantiomers was discovered to have different effects for in vivo nucleic acid delivery including siRNA. We have found that using S-DOTAP, one of two enantiomer of DOTAP, is more effective for in vitro siRNA transfection and in vivo (respiratory track) siRNA delivery. Therefore, we have designated S-DOTAP as Snano-5 for potential siRNA delivery into ocular tissue.

We have tested spermine and spermidine based material for in vitro and in vivo siRNA delivery in multiple cell types and mouse tissue types. One form of the spermine based system is efficient for human A549 cell siRNA transfection and respiratory track siRNA delivery. We designate it as Snano-6.

### Example 17

### Chemical Modification of siRNA for Ocular Therapeutics

Single-stranded nucleic acids are rapidly degraded in serum or inside cells. Double-stranded nucleic acids, including siRNAs, are more stable than their single-stranded counterparts, but are still degraded and must be protected from nuclease attack if use includes exposure to serum. Protection can be provided externally through use of a suitable delivery tool (such as complexation with a nanoparticle or encapsulation within a liposome as we described in Example 16) or intrinsically through use of nuclease resistant chemical modification of the nucleic acid itself. The simplest approach to increase nuclease stability is to directly modify the internucleotide phosphate linkage. Replacement of a non-bridging oxygen with sulfur (PS), boron (boranophosphate), nitrogen (phosphoramidate), or methyl (methylphosphonate) groups will provide nuclease resistance and have all been used to help stabilize single-stranded antisense oligonucleotides.

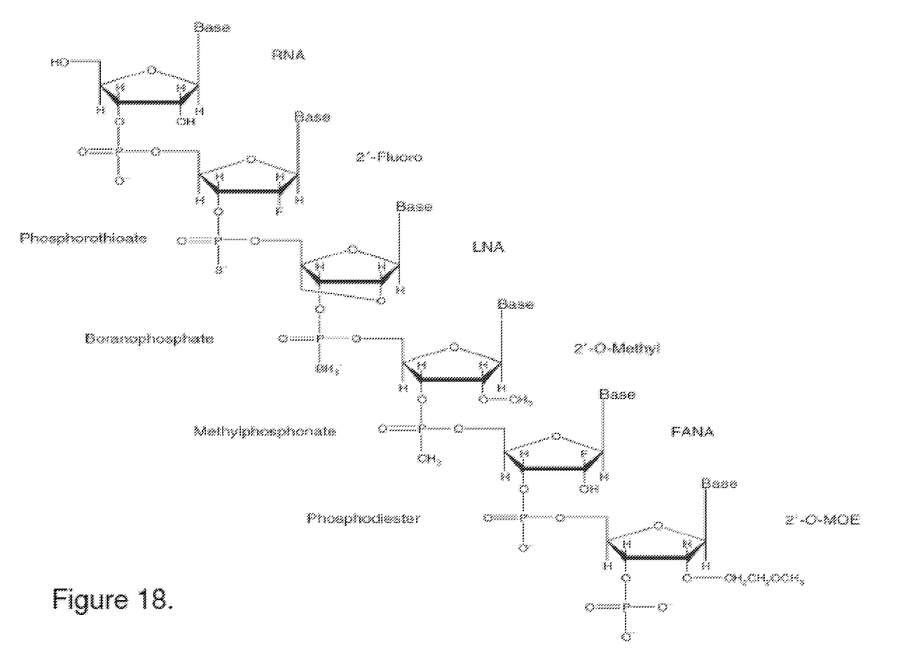

FIG. 18 shows various modifications that improve nuclease stability and can be employed in siRNAs. Phosphoramidate and methylphosphonate derivatives were extensively explored for use in antisense applications and were found to significantly alter interactions between the nucleic acid and cellular enzymes, such as RNase H. Their use has not been systematically studied for use in RNAi. Boranophosphate modified DNA or RNA is resistant to nuclease degradation and the boron modification appears to be compatible with siRNA function; however, boranophosphates are not easily made using chemical synthesis. The PS modification is easily made and has been extensively used to improve nuclease stability of both antisense oligonucleotides and siRNAs.

Phosphorothioate modified nucleic acids are sulfated polyanions that are “sticky” and can nonspecifically bind to a variety of cellular proteins, potentially causing unwanted side effects. Nevertheless, this modification can be safely used to improve stability of a siRNA. Restricting placement of PS-modified bonds to the ends of the oligonucleotides will provide resistance to exonucleases while minimizing the overall PS content of the oligo, thereby limiting unwanted side effects. Given the long history of use of PS-modi” ed antisense oligonucleotides, the potential toxicity of this modification is well understood and PS-modified compounds can be safely administered.

Modification of the 2-position of the ribose can indirectly improve nuclease resistance of the internucleotide phosphate bond and at the same time can increase duplex stability (Tm) and may also provide protection from immune activation. 2-O-methyl RNA (2 OMe) is a naturally occurring RNA variant found in mammalian ribosomal RNAs and transfer RNAs. It is nontoxic and can be placed within either the S or AS strands of a siRNA.

The 2 fluoro (2-F) modification is compatible with siRNA function and also helps stabilize the duplex against nuclease degradation. Incorporation of 2-F at pyrimidine positions maintains siRNA activity in vitro and in vivo. The 2-F modification is even tolerated at the site of Ago2 cleavage. The combined use of 2-F pyrimidines with 2 OMe purines can results in RNA duplexes with extreme stability in serum and improved in vivo performance.

Locked nucleic acids (LNAs) contain a methylene bridge which connects the 2-O with the 4-C of the ribose. The methylene bridge “locks” the sugar in the 3-endo conformation, providing both a signi” cant increase in Tm as well as nuclease resistance. Extensive modification of a siRNA with LNA bases generally results in decreased activity (even more so than 2 OMe); however, siRNAs with limited incorporation retain functionality and offer significant nuclease stabilization.

The 2 OMe modification is a naturally occurring RNA variant and its use in synthetic siRNAs is not anticipated to present significant toxicity. Other 2-modifications discussed here are not naturally occurring and their potential for toxic side effects needs to be considered. The 2-F modification has been studied for safety as a component of synthetic oligonucleotides.

The modification strategies discussed above are intended to impart nuclease resistance to 21-mer siRNAs while retaining the ability of the duplexes to enter RISC and maintain guide-strand function with Ago2. For our ocular siRNA therapeutics using 25-mer blunt ended siRNA, we will combine strategy using nanoparticle carrier with somewhat chemical modification to improve the therapeutic benefit while minimize the potential toxicity, and complications at the later product manufacturing stage.

### Example 18

### Combination siRNA Therapeutics with Other Ongoing Therapeutics

There several therapeutic agents are being used in the clinic, including the monoclonal antibody drugs (Avastin and Lucentis), soluble receptor agent (VEGF trap) and others. Because of different mechanisms of action we are using with siRNA drugs, blocking the production of VEGF and VEGFR2 production rather than blocking their function, the combined regimen can be expected. In our attempt using siRNA with a monoclonal antibody drug in the same regimen, we have observed a clear improvement of the therapeutic benefit (data is not shown). In our siRNA therapeutic for ocular neovascularization conditions, the combined use of both siRNA and other inhibitors will be expected. The latest research has identified that a number of micro RNA (miRNA) are also involved in the antiogenesis of various neovascularization diseases, such as mir-132 can serve as a drug target for anti-mir or siRNA inhibitors to reverse the pathological conditions. Combination of siRNA and anti-mir inhibitors also represent a novel approach for ocular angiogenesis conditions.

### Example 19

### HKP-siRNA Drug Development Process

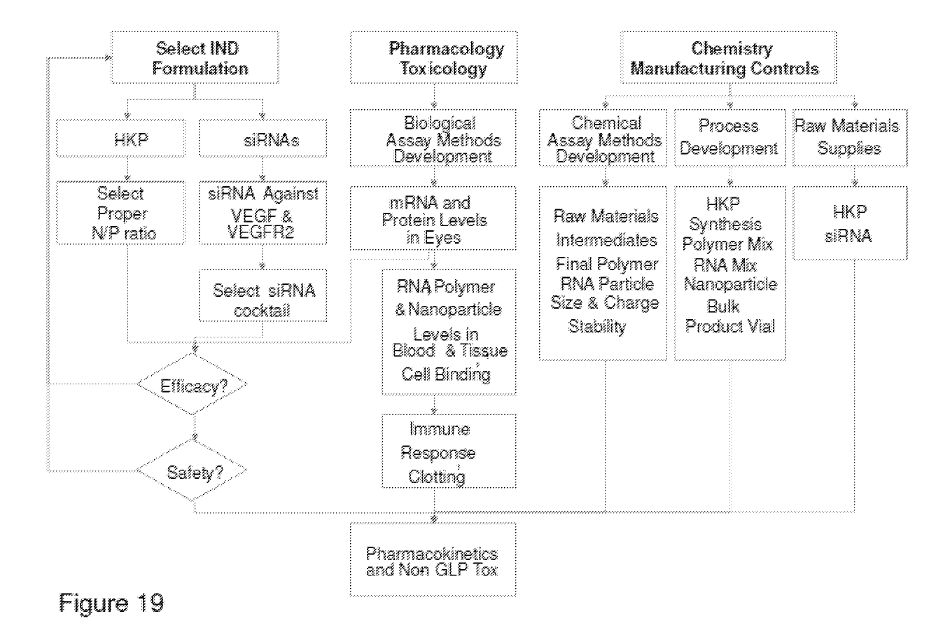

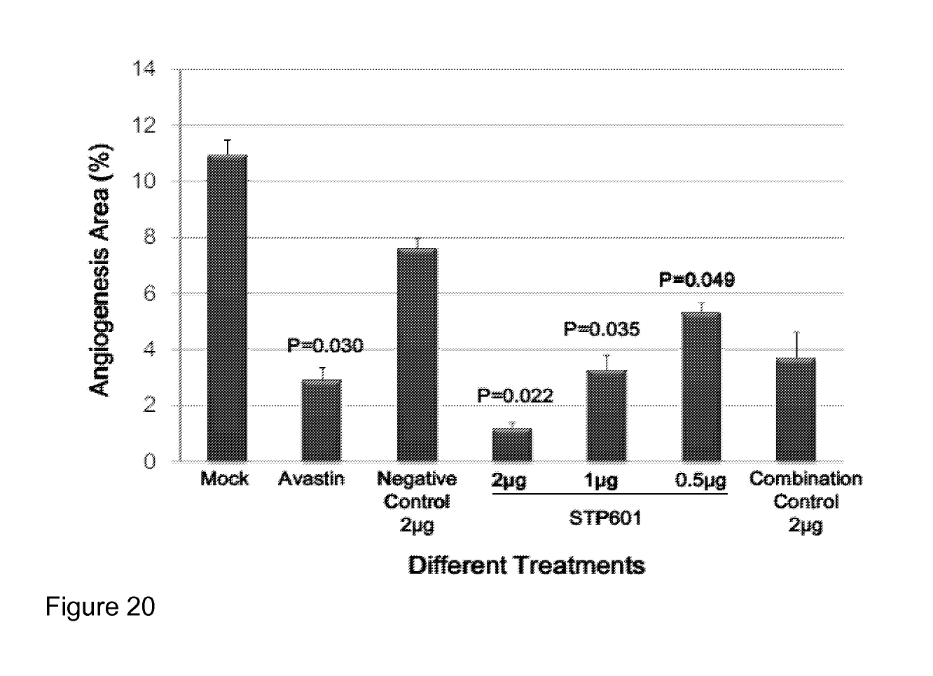

Drug development process for siRNA therapeutics is going to be somewhat different from other therapeutic modalities like small molecule and protein drugs. We have developed a process for HKP-siRNA therapeutics production and manufacture, followed by a series of pharmacology and toxicology characterizations, in order to meet the requirements from the regulatory bodies for drug approval (FIG. 19).

### Example 20

### Comparing Avastin with the STP601 siRNA Therapeutic Using Mouse ROP Model

We have evaluate the therapeutic potential of our siRNA therapeutic product, STP601, which consists of siRNA duplexes targeting VEGF and VEGFR2 and packaged with HKP using the mouse retinopathy of prematurity (ROP) model. The total number of mice used in the study was 120. The retinal NV in ROP model was induced by hyperoxia. Briefly, the pups with the nursing dams were maintained in hyperoxia environment (75%+2 oxygen) from postnatal days P7 to P12, then returned to room air (normoxia). On day 12^{th} post birth, we divided the neonatal mice into 7 groups: 1). Control group without any treatment; 2). Positive control group with single dose of Avastin at 25 μg/eye; 3). Negative control using single dose of non-related siRNA at 2 μg/eye; 4). Testing group 1 with single dose of STP601 at 2 μg/eye; 5). Testing group 2 with single dose of STP601 at 1 μg/eye; 6). Testing group 3 with single dose of STP601 at 0.5 μg/eye; and 7). Testing group 4 with single dose of different siRNA combination targeting VEGF and TGFβ1 at 2 μg/eye.

Before the drug treatment, the mice were anesthetized with Avertin solution according to the IACUC certified SOP. The agents were intra-vitreously injected into the right side of eye of each mouse. On day 17 post birth, 0.5 ml of 50 mg/ml of FITC-Dextran solution was injected into each mouse. Then the mouse eye samples were collected and analyzed. The angiogenesis areas of each eye of each mouse were measured and compared. As shown in FIG. 21, STP601 testing samples, consisting of two siRNA duplexes targeting both VEGF and VEGFR2 mRNA, exhibited clear therapeutic benefit with this ROP mouse model, which is even better than the Avastin treatment with much higher dosage, 6-8 mice per group were tested.

TABLE 1
Selection of potent siRNA targeting VEGF:

| | | |
|---|---|---|
| hmVEGFa | Sense: | 5′-r(CCAUGCCAAGUGGUCCCAGGCUGCA)-3′ |
| | Anti- | 5′-r(UGCAGCCUGGGACCACUUGGCAUGG)-3′ |
| | sense: | |
| hmVEGFb | Sense: | 5′-r(CCAACAUCACCAUGCAGAUUAUGCG)-3′ |
| | Anti- | 5′-r(CGCAUAAUCUGCAUGGUGAUGUUGG)-3′ |
| | sense: | |
| hmVEGFc | Sense: | 5′-r(CUGUAGACACACCCACCCACAUACA)-3′ |
| | Anti- | 5′-r(UGUAUGUGGGUGGGUGUGUCUACAG)-3′ |
| | sense: | |
| hmVEGFd | Sense: | 5′-r(CACUUUGGGUCCGGAGGGCGAGACU)-3′ |
| | Anti- | 5′-r(AGUCUCGCCCUCCGGACCCAAAGUG)-3′ |
| | sense: | |
| hmVEGFe: | Sense: | 5′-r(CCUGAUGAGAUCGAGUACAUCUUCA)-3′ |
| | Anti- | 5′-r(UGAAGAUGUACUCGAUCUCAUCAGG)-3′ |
| | sense: | |
| hmVEGFf: | Sense: | 5′-r(GAGAGAUGAGCUUCCUACAGCACAA)-3′ |
| | Anti- | 5′-r(UUGUGCUGUAGGAAGCUCAUCUCUC)-3′ |
| | sense: | |
| hmVEGFg: | Sense: | 5′-r(GCAAGGCGAGGCAGCUUGAGUUAAA)-3′ |
| | Anti- | 5′-r(UUUAACUCAAGCUGCCUCGCCUUGC)-3′ |
| | sense: | |
| hmVEGFh: | Sense: | 5′-r(CACAACAAAUGUGAAUGCAGACCAA)-3′ |
| | Anti | 5′-r(UUGGUCUGCAUUCACAUUUGUUGUG)-3′ |
| | sense: | |

TABLE 2
Selection of potent siRNA targeting VEGFR2:

| | | |
|---|---|---|
| hVR2a: | Sense: | 5′-r(CCUCUUCUGUAAGACACUCACAAUU)-3′ |
| | Anti- | 5′-r(AAUUGUGAGUGUCUUACAGAAGAGG)-3′. |
| | sense: | |
| hVR2b: | Sense: | 5′-r(CCCUUGAGUCCAAUCACACAAUUAA)-3′ |
| | Anti- | 5′-r(UUAAUUGUGUGAUUGGACUCAAGGG)-3′ |
| | sense: | |
| hVR2c: | Sense: | 5′-r(CCAAGUGAUUGAAGCAGAUGCCUUU)-3′ |
| | Anti- | 5′-r(AAAGGCAUCUGCUUCAAUCACUUGG)-3′ |
| | sense: | |
| hmVR2d: | Sense: | 5′-r(GAGCAUGGAAGAGGAUUCUGGACUC)-3′ |
| | Anti- | 5′-r(GAGUCCAGAAUCCUCUUCCAUGCUC)-3′ |
| | sense: | |
| hmVR2e: | Sense: | 5′-r(CAUGGAAGAGGAUUCUGGACUCUCU)-3′ |
| | Anti- | 5′-r(AGAGAGUCCAGAAUCCUCUUCCAUG)-3′ |
| | sense: | |
| hmVR2f: | Sense: | 5′-r(CCUGACCUUGGAGCAUCUCAUCUGU)-3′ |
| | Anti- | 5′-r(ACAGAUGAGAUGCUCCAAGGUCAGG)-3′ |
| | sense: | |
| hmVR2g: | Sense: | 5′-r(GCUAAGGGCAUGGAGUUCUUGGCAU)-3′ |
| | Anti- | 5′-r(AUGCCAAGAACUCCAUGCCCUUAGC)-3′ |
| | sense: | |
| hmVR2h: | Sense: | 5′-r(GACUUCCUGACCUUGGAGCAUCUCA)-3′ |
| | Anti- | 5′-r(UGAGAUGCUCCAAGGUCAGGAAGUC)-3′ |
| | sense: | |

TABLE 3
Selection of potent siRNA targeting TGFβ1:

| | | |
|---|---|---|
| hmTFb1a: | Sense | 5′-r(GGAUCCACGAGCCCAAGGGCUACCA)-3′ |
| | Anti- | 5′-r(UGGUAGCCCUUGGGCUCGUGGAUCC)-3′ |
| | sense | |
| hmTFb1b: | Sense | 5′-r(CCCAAGGGCUACCAUGCCAACUUCU)-3′ |
| | Anti- | 5′-r(AGAAGUUGGCAUGGUAGCCCUUGGG)-3′ |
| | sense | |
| hmTFb1c: | Sense | 5′-r(GAGCCCAAGGGCUACCAUGCCAACU)-3′ |
| | Anti- | 5′-r(AGUUGGCAUGGUAGCCCUUGGGCUC)-3′ |
| | sense | |
| hmTF25d: | Sense | 5′-r(GAUCCACGAGCCCAAGGGCUACCAU)-3′ |
| | Anti- | 5′-r(AUGGUAGCCCUUGGGCUCGUGGAUC)-3′ |
| | sense | |
| hmTF25e: | Sense | 5′-r(CACGAGCCCAAGGGCUACCAUGCCA)-3′ |
| | Anti- | 5′-r(UGGCAUGGUAGCCCUUGGGCUCGUG)-3′ |
| | sense | |
| hmTF25f: | Sense | 5′-r(GAGGUCACCCGCGUGCUAAUGGUGG)-3′ |
| | Anti- | 5′-r(CCACCAUUAGCACGCGGGUGACCUC)-3′ |
| | sense | |
| hmTF25g: | Sense | 5′-r(GUACAACAGCACCCGCGACCGGGUG)-3′ |
| | Anti- | 5′-r(CACCCGGUCGCGGGUGCUCUUCUAC)-3′ |
| | sense | |
| hmTF25h: | Sense | 5′-r(GUGGAUCCACGAGCCCAAGGGCUAC)-3′ |
| | Anti- | 5′-r(GUAGCCCUUGGGCUCGUGGAUCCAG)-3 |
| | sense | |

### REFERENCES

1. Folkman, J, 1971: Tumor Angiogenesis: Therapeutic Implications. N. Engl. J. Med., Vol. 285, p1182-1186.
2. Leung, D. W., Cachianes, G., Kuang, W. J., Goeddel, D. V., and Ferrara, N., 1989: Vascular Endothelial Growth Factor is a Secreted Angiogenic Mitogen. Science, Vol. 246, p1306-1309.
3. Aiello, L. P. et al. 1995: Suppression of Retinal Neovascularization in vivo by Inhibition of Vascular Endothelial Growth Factor (VEGF) Using Soluble VEGF-Receptor Chimeric Proteins. PNAS, Vol. 92, pp. 10457-10461.
4. Alice L. Yu, Rudolf Fuchshofer, et al. 2009. Subtoxic Oxidative Stress Induces Senescence in Retinal Pigment Epithelial Cells via TGF-Release 1,4 Investigative Ophthalmology & Visual Science, Vol. 50, No. 2
5. Elbashir, S. M., Lendeckel, W. and Tuschl, T. 2001: RNA interference is mediated by 21- and 22-nucleotide RNAs. Genes Dev. 15(2):188-200.
6. Lu, Patrick Y., Frank Y. Xie and Martin Woodle, (2003): SiRNA-Mediated Antitumorigenesis For Drug Target Validation And Therapeutics. Current Opinion in Molecular Therapeutics, 5(3):225-234.
7. Mark A. Behlke, Chemical Modification of siRNAs for In Vivo Use. Oligonucleotides 18:305-320 (2008)
8. Semizarov, D., L. Frost, A. Sarthy, P. Kroeger, D. Halbert and S. W. Fesik, 2003: Specificity of Short Interfering RNA Determined Through Gene Expression Signatures. PNAS. 1131959100.
9. Hough, S. R., K. A. Wiederholt, A. C. Burlier, T. M. Woolf and M. F. Taylor, 2003: Why RNAi Makes Sense. Nature Biotechnology, 21(7) 731-732.
10. Lu, Patrick, Frank Xie, Quinn Tang, Jun Xu, Puthupparampil Scaria, Qin Zhou and Martin Woodle, 2002: Tumor Inhibition By RNAi-Mediated VEGF and VEGFR2 Down Regulation in Xenograft Models. Cancer Gene Therapy, Vol. 10, Supplement 1, 011.
11. Kleinman M E, et al. 2008. Sequence- and target-independent angiogenesis suppression by siRNA via TLR3. Nature. 452(7187):591-7.
12. Xie, F. Y. Lu, P. Y. et al. 2002: Methods of In Vivo Down Regulation of Targeted Gene Expression By Introduction of RNA Interference. U.S. patent application, 60/401,029
13. Mei Zheng, Dennis M. Klinman†, Malgorzata Gierynska, and Barry T. Rouse, 2002: DNA containing CpG motifs induces angiogenesis. PNAS, Vol. 99(13).
14. International Patent Application No. PCT/US2008/066298, filed Jun. 9, 2008, and published on Dec. 18, 2008 as International Publication No. WO 2008/154482 A2.
15. U.S. Provisional Patent Application No. 60/942,898 filed Jun. 8, 2007.

All publications, including issued patents and published patent applications, all database entries identified by url addresses or accession numbers, and all U.S. patent applications, whether or not published, are incorporated herein by reference in their entireties.

Although this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A composition comprising at least two different siRNA duplexes and a pharmaceutically acceptable carrier, wherein one of said siRNA duplexes binds to an mRNA molecule that encodes VEGF and the other of said siRNA duplexes binds to an mRNA molecule that encodes VEGFR2.

2. The composition of claim 1 further comprising an siRNA duplex that binds to an mRNA molecule that encodes TGFβ1.

3. The composition of claim 1 wherein said siRNA duplexes target both human mRNA and homologous mouse mRNA.

4. The composition of claim 2 wherein said siRNA duplexes target both human mRNA and homologous mouse mRNA.

5. The composition of claim 1 wherein said siRNA duplexes comprise oligonucleotides with a length of 16-27 base pairs.

7. The composition of claim 1 wherein said siRNA duplexes comprise oligonucleotides with a length of 21-25 base pairs.

8. The composition of claim 1 wherein said siRNA duplexes comprises oligonucleotides with a length of 25 base pairs.

9. The composition of claim 8 wherein said siRNA duplexes comprise oligonucleotides with blunt ends at both ends.

10. The composition of claim 1 wherein said siRNA duplexes are selected from the siRNA duplexes listed in Tables 1 and 2.

11. The composition of claim 2 wherein said siRNA duplexes are selected from the siRNA duplexes in Tables 1-3.

12. The composition of claim 1 comprising the siRNA duplex hmVEGFc:

| (SEQ ID NO: 6) | |
|---|---|
| Sense: | 5′-CUGUAGACACACCCACCCACAUACA-3′, |
| (SEQ ID NO: 20) | |
| Antisense: | 5′-UGUAUGUGGGUGGGUGUGUCUACAG-3′ |

and
the siRNA duplex hmVR2h:

| (SEQ ID NO: 7) | |
|---|---|
| Sense, | 5′-GACUUCCUGACCUUGGAGCAUCUCA-3′, |
| (SEQ ID NO: 43) | |
| Antisense, | 5′-UGAGAUGCUCCAAGGUCAGGAAGUC-3′. |

13. The composition of claim 2 comprising the siRNA duplex hmVEGFc:

| (SEQ ID NO: 6) | |
|---|---|
| Sense, | 5′-CUGUAGACACACCCACCCACAUACA-3′, |
| (SEQ ID NO: 20) | |
| Antisense, | 5′-UGUAUGUGGGUGGGUGUGUCUACAG-3′, |

the siRNA duplex hmVR2h:

| (SEQ ID NO: 7) | |
|---|---|
| Sense, | 5′-GACUUCCUGACCUUGGAGCAUCUCA-3′, |
| (SEQ ID NO: 43) | |
| Antisense, | 5′-UGAGAUGCUCCAAGGUCAGGAAGUC-3′, |

and
the siRNA duplex hmTF25f:

| (SEQ ID NO: 8) | |
|---|---|
| Sense, | 5′-GAGGUCACCCGCGUGCUAAUGGUGG-3′, |
| (SEQ ID NO: 54) | |
| Antisense, | 5′-CCACCAUUAGCACGCGGGUGACCUC-3′. |

14. The composition of claim 1 wherein said duplexes are selected from the group consisting of:
a. derived duplexes consisting of 24 contiguous base pairs of any one or more of the duplexes in Tables 1 and 2;
b. derived duplexes consisting of 23 contiguous base pairs of any one or more of the duplexes in Tables 1 and 2;
c. derived duplexes consisting of 22 contiguous base pairs of any one or more of the duplexes in Tables 1 and 2;
d. derived duplexes consisting of 21 contiguous base pairs of any one or more of the duplexes in Tables 1 and 2;
e. derived duplexes consisting of 20 contiguous base pairs of any one or more of the duplexes in Tables 1 and 2;
f. derived duplexes consisting of 19 contiguous base pairs of any one or more of the duplexes in Tables 1 and 2;
g. derived duplexes consisting of 18 contiguous base pairs of any one or more of the duplexes in Tables 1 and 2;
h. derived duplexes consisting of 17 contiguous base pairs of any one or more of the duplexes in Tables 1 and 2; and
i. derived duplexes consisting of 16 contiguous base pairs of any one or more of the duplexes in Tables 1 and 2.

15. The composition of claim 2 wherein said duplexes are selected from the group consisting of:
a. derived duplexes consisting of 24 contiguous base pairs of any one or more of the duplexes in Tables 1-3;
b. derived duplexes consisting of 23 contiguous base pairs of any one or more of the duplexes in Tables 1-3;
c. derived duplexes consisting of 22 contiguous base pairs of any one or more of the duplexes in Tables 1-3;
d. derived duplexes consisting of 21 contiguous base pairs of any one or more of the duplexes in Tables 1-3;
e. derived duplexes consisting of 20 contiguous base pairs of any one or more of the duplexes in Tables 1-3;
f. derived duplexes consisting of 19 contiguous base pairs of any one or more of the duplexes in Tables 1-3;
g. derived duplexes consisting of 18 contiguous base pairs of any one or more of the duplexes in Tables 1-3;
h. derived duplexes consisting of 17 contiguous base pairs of any one or more of the duplexes in Tables 1-3; and
i. derived duplexes consisting of 16 contiguous base pairs of any one or more of the duplexes in Tables 1-3.

16. The composition of claim 1 further comprising an additional compound that inhibits neovascularization in the eye of a subject.

17. The composition of claim 1 wherein said carrier is selected from the group consisting of a branched peptide, a polymer, a lipid, and a micelle.

18. The composition of claim 1 wherein said carrier comprises a histidine-lysine co-polymer.

19. The composition of claim 1 wherein said composition comprises a nanoparticle.

20. A method for treating ocular disease in a subject, wherein said disease is characterized at least in part by neovascularization, comprising administering to said subject a therapeutically effective amount of the composition of claim 1.

21. A method for treating ocular disease in a subject, wherein said disease is characterized at least in part by neovascularization, comprising administering to said subject a therapeutically effective amount of the composition of claim 2.

22. The method of claim 20 wherein said ocular disease is in the retina of the eye.

23. The method of claim 20 where the ocular disease is selected from the group consisting of proliferative diabetic retinopathy, macular edema, and age-related macular degeneration.

24. The method of claim 20 wherein the subject is a mammal.

25. The method of claim 20 wherein the subject is a human.
